(19) United States
(12) Patent Application Publication
(10) Pub. No.: US 2026/0354766 A1
(43) Pub. Date: Nov. 12, 2026

(54) Multimodal Physiological Monitoring and Neurofeedback System With Sensor-driven Closed-loop Intervention Delivery

(71) Applicant: Optios, Inc., Greenville, SC (US)

(72) Inventors: Jacek Dmochowski, Montclair, NJ (US); Josef Faller, Barcelona (ES); James Orme-Rogers, New York, NY (US); Derek Netto, Jamaica, NY (US); Paul Sajda, Westport, CT (US); Paul DeGuzman, New York, NY (US); Alec Pierce, Puyallup, WA (US); David Bach, Carlsbad, CA (US); David Sammons, Travelers Rest, SC (US); Jesse Wright, Drexel Hill, PA (US)

(21) Appl. No.: 19/650,212

(22) Filed: Apr. 16, 2026

Related U.S. Application Data
(63) Continuation of application No. PCT/US2026/023786, filed on Apr. 15, 2026.
(60) Provisional application No. 63/789,211, filed on Apr. 15, 2025.
(60) Provisional application No. 63/789,175, filed on Apr. 15, 2025.
(60) Provisional application No. 63/841,344, filed on Jul. 9, 2025.

(51) Int. Cl. A61B 5/00; A61B 5/0205; A61B 5/08; A61B 5/11; A61B 5/16; A61B 5/318; A61B 5/369; G06F 40/40

(52) U.S. Cl. CPC A61B 5/7203; A61B 5/0205; A61B 5/11; A61B 5/163; A61B 5/318; A61B 5/369; G06F 40/40; A61B 5/08; A61B 2503/10; A61B 2503/22

(57) ABSTRACT

A closed-loop physiological control method for physiological monitoring and biofeedback delivery includes obtaining physiological signals (electroencephalography signals, electrocardiography signals, eye-tracking signals, respiratory signals, or biomechanical motion signals) from physiological sensors associated with an operator during performance of a task. The method includes conditioning the physiological signals to remove artifacts, including independent component analysis for removing eye movement artifacts and muscle noise, bandpass filtering, or notch filtering. The method includes processing the physiological signals through a first model to generate physiological state embeddings. The method includes projecting the physiological state embeddings into a token space of a second model to produce projected embeddings. The method includes determining, using the second model operating on the projected embeddings and contextual information about the operator, a neurophysiological state of the operator. The method includes selecting, based on the neurophysiological state, a biofeedback stimulus and delivering the biofeedback stimulus to the operator.

### CROSS-REFERENCE TO RELATED APPLICATIONS

This application is a continuation of PCT/US2026/023786 filed Apr. 15, 2026, which claims the benefit of U.S. Provisional Application Nos. 63/789,175 filed Apr. 15, 2025, 63/789,211 filed Apr. 15, 2025, and 63/841,344 filed Jul. 9, 2025. The entire disclosures of the above applications are incorporated by reference.

### FIELD

The present disclosure relates to physiological monitoring and neurofeedback systems and, more particularly, to a multimodal sensing apparatus that simultaneously acquires physical signals from a subject, processes the acquired signals to determine the subject's neurophysiological state, and delivers closed-loop biofeedback interventions to the subject.

### BACKGROUND

Human performance in high-stakes environments, such as aviation, military operations, elite athletics, financial trading, competitive gaming, surgical medicine, and in knowledge work environments such as software development, data analysis, research, and document authoring, depends critically on the cognitive and physiological state of the operator. Fatigue, cognitive overload, attention lapses, stress, and emotional dysregulation degrade decision-making capacity well before behavioral indicators become apparent. Conventional performance monitoring systems rely primarily on observable outputs: actions taken, response times, error rates, and post-hoc outcome analysis. Such systems are fundamentally blind to the internal physiological dynamics that causally drive performance degradation.

Additionally, existing physiological monitoring approaches suffer from several limitations. First, traditional brain-computer interface (BCI) and biofeedback systems rely on handcrafted feature extraction pipelines that require domain-specific expertise to design and are brittle across tasks, individuals, and sensor configurations. Second, these systems typically operate in isolation, analyzing individual physiological modalities without the benefit of multimodal fusion. Third, conventional systems lack contextual reasoning—they can detect statistical anomalies but cannot understand what the operator is doing, why a particular physiological pattern is significant, or what intervention would be appropriate.

In the domain of sports performance analysis, existing systems, such as ball-tracking technologies and computer vision systems, analyze observable physical movements and outcomes without insight into the athlete's internal cognitive and physiological states. For example, electroencephalography (EEG) neurofeedback systems may monitor brain activity but fail to correlate it with actual performance outcomes. Similarly, heart rate variability monitors track autonomic function but lack integration with motor performance data. No existing sports technology system provides a comprehensive understanding of the relationship between an athlete's neurophysiological state, behavioral patterns, and performance outcomes.

The emergence of large language models (LLMs) has created powerful general-purpose reasoning engines, but these models operate exclusively in the domain of natural language and have no native capacity to process real-time physiological signals. Current approaches to incorporating physiological data into LLM-based systems are limited to crude text-based summaries, discarding the rich temporal dynamics, spectral structure, and cross-modal correlations essential for accurate state estimation.

Furthermore, existing performance optimization systems operate myopically—selecting the next intervention based on the current state without considering how that intervention will reshape physiological dynamics over time. Real-world performance optimization requires trajectory-aware policies that account for temporal dynamics and optimize for long-horizon outcomes.

Additionally, there is an absence of scalable supervision mechanisms for training performance models. Expert human evaluation of decision quality is expensive, slow, and does not scale. Performance models are trained on sparse outcome signals rather than dense, context-aware assessments of decision quality.

Finally, no existing system provides comprehensive frameworks for: (a) neurotyping (the characterization of stable individual-specific physiological response patterns); (b) cognitive primitive extraction (the identification of fundamental cognitive building blocks underlying performance); (c) automated biomarker discovery (the systematic identification and validation of novel physiological markers correlated with performance); or (d) multi-timescale feedback delivery spanning immediate haptic cues through long-term training program optimization. Additionally, no existing system provides: (e) observation of the operator's task environment through visual captures and behavioral telemetry to provide contextual grounding for physiological state assessments; (f) structured interface protocols that enable external software agents to modify their behavior based on the operator's cognitive state without requiring domain expertise in physiological signal interpretation; or (g) longitudinal user modeling that accumulates operator decisions, preferences, and physiological responses to serve as the operator's digital cognitive representative across an ecosystem of connected software agents.

Further, there is an absence of systems that observe the operator's task environment, such as screen content, application context, and visible error states, and combine these observations with physiological and behavioral signals to assess cognitive performance in context. Existing AI coding assistants and AI agents have no awareness of the operator's cognitive state. No existing system combines multimodal machine learning model analysis of visual captures of the task environment with physiological sensing and behavioral telemetry to produce contextually-grounded cognitive performance assessments and deliver targeted interventions.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

One aspect of the present disclosure provides a method including obtaining sensor data from a set of physiological sensors associated with an operator during performance of a task and removing artifacts from the sensor data. The method further includes converting the sensor data, using a foundation model, into a set of physiological state embeddings and projecting the set of physiological state embeddings into a token space of a large language model (LLM). The method further includes generating an intervention for the operator, using the LLM, based on the set of physiological state embeddings and additional information about the task, and delivering the intervention to the operator.

Another aspect of the present disclosure provides a system including a set of physiological sensors, data processing hardware communicatively coupled to the set of physiological sensors, and a user device. The set of physiological sensors is configured to obtain sensor data of an operator during performance of a task. The data processing hardware is operable to receive the sensor data and remove artifacts from the sensor data. The data processing hardware is operable to convert, using a foundation model, the sensor data into a set of physiological state embeddings and project the set of physiological state embeddings into a token space of a large language model (LLM). The data processing hardware is operable to generate, using the LLM, an intervention for the operator based on the set of physiological state embeddings and additional information about the task and send the intervention to the operator. The user device is configured to receive the intervention and deliver the intervention to the operator.

Another aspect of the present disclosure relates to systems that observe an operator's task environment through visual captures processed by multimodal machine learning models, integrate behavioral telemetry from operator interactions including keyboard, pointing device, and version control activity, align gaze-tracking data with visually observed task content to detect comprehension difficulty, and expose pre-interpreted cognitive state intelligence through structured interface protocols that enable external software agents to modify their behavior based on the operator's current cognitive state. The disclosure is applicable to high-stakes operational and competitive domains and knowledge work environments including software development, data analysis, research, and document authoring.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a block diagram illustrating an overall system architecture of a physiology-aware intelligence platform including multimodal signal acquisition, foundation model, vector embedding projection, LLM reasoning, closed-loop intervention, and evaluation subsystems according to the principles of the present disclosure.

FIG. 2 is a block diagram illustrating a multimodal physiological signal acquisition subsystem including EEG, ECG, eye-tracking, respiratory, and biomechanical channels with artifact removal and preprocessing pipelines according to the principles of the present disclosure.

FIG. 3 is a block diagram illustrating a neuroperformance foundation model architecture including self-supervised learning objectives, temporal encoding, and multi-resolution feature extraction according to the principles of the present disclosure.

FIG. 4 is a block diagram illustrating the embedding projection layer (Vector-ICL) mapping physiological embeddings into large language model token space for native reasoning according to the principles of the present disclosure.

FIG. 5 is a block diagram illustrating a closed-loop intervention subsystem including state estimation, intervention selection, and compliance scoring according to the principles of the present disclosure.

FIG. 6 is a block diagram illustrating an AI Judge evaluation subsystem for scalable assessment of decision quality using large language models according to the principles of the present disclosure.

FIG. 7 is a block diagram illustrating a neurotyping subsystem including feature extraction, archetype classification, and individual profile characterization according to the principles of the present disclosure.

FIG. 8 is a block diagram illustrating a trajectory-aware policy optimization module accounting for multi-step dynamics and long-horizon performance outcomes according to the principles of the present disclosure.

FIG. 9 is a flowchart illustrating a real-time inference pipeline from signal acquisition through feedback delivery according to the principles of the present disclosure.

FIG. 10 is a block diagram illustrating a platform API architecture exposing physiological intelligence to external applications via REST, streaming, and machine-readable protocols according to the principles of the present disclosure.

FIG. 11 is a block diagram illustrating a cognitive primitive extraction module, including focused visual attention, motor preparation, flow state detection, and cognitive load assessment pipelines according to the principles of the present disclosure.

FIG. 12 is a block diagram illustrating a biomarker library and discovery subsystem, including a priori biomarker storage, data-driven biomarker discovery, validation, and integration into real-time monitoring according to the principles of the present disclosure.

FIG. 13 is a block diagram illustrating a visualization engine and user interface subsystem, including real-time performance dashboards, interactive data exploration, and multi-format export capabilities according to the principles of the present disclosure.

FIG. 14 is a flowchart illustrating a sport-specific implementation of the system for basketball free throw optimization, including pre-shot routine analysis, zone state detection, and intervention protocol selection according to the principles of the present disclosure.

FIG. 15 is a block diagram illustrating a visual and behavioral observation subsystem, including a visual capture module, a hierarchical multimodal machine learning pipeline, a gaze-content alignment module, a behavioral telemetry module, a multimodal fusion module, and a privacy-preserving embedding extraction module according to the principles of the present disclosure.

FIG. 16 is a flowchart illustrating a gaze-content alignment process, including fixation coordinate mapping onto semantic regions of a visual capture, comprehension difficulty detection from persistent attentional return patterns, and content-specific intervention triggering according to the principles of the present disclosure.

FIG. 17 is a block diagram illustrating a structured interface protocol architecture, including pre-interpreted behavioral guidance functions, a bidirectional communication channel, and user preference prediction according to the principles of the present disclosure.

FIG. 18 is a block diagram illustrating a user modeling subsystem, including episodic memory, a semantic knowledge model, decision-outcome accumulation, vector representation storage, preference learning, predictive action modeling, and digital cognitive representative functions according to the principles of the present disclosure.

In the drawings, reference numbers may be reused to identify similar and/or identical elements.

### DETAILED DESCRIPTION

### System Overview and Architecture

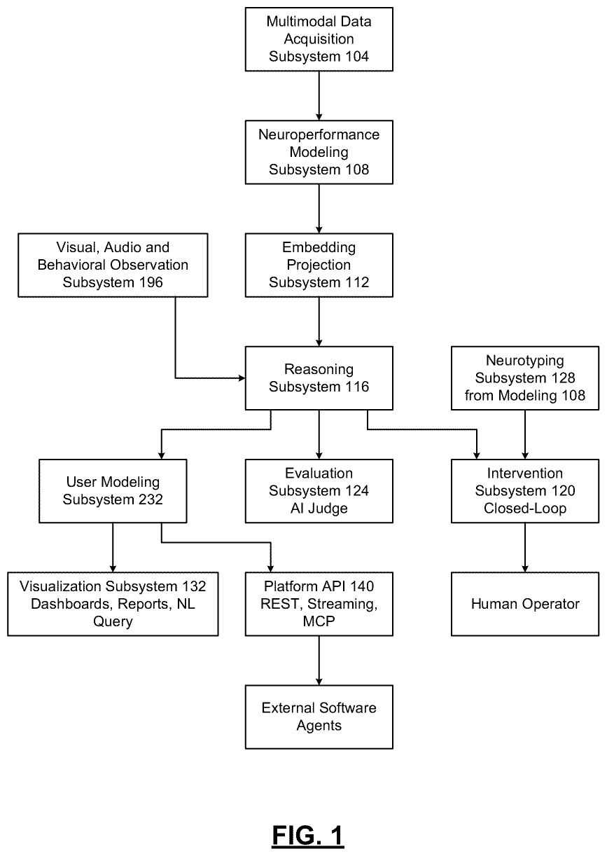

FIG. 1 illustrates a platform 100. In various implementations, the platform 100 is a physiology-aware intelligence platform. The platform 100 may include various interconnected subsystems operating in concert to enable real-time performance assessment, prediction, biofeedback, optimization, and intervention of an operator (not illustrated) performing a task. In various implementations, the operator repeatedly performs the task. As used herein, “real-time” may refer to processing with latency sufficiently low to enable feedback or intervention during ongoing performance, typically less than one second from measurement to feedback delivery. As used herein, “operator” may refer to any person whose cognitive performance is being assessed, predicted, or optimized by the platform 100.

The platform 100 may include a multimodal data acquisition subsystem 104, a neuroperformance modeling subsystem 108, an embedding projection subsystem 112, a reasoning subsystem 116, an intervention subsystem 120, an evaluation subsystem 124, a neurotyping subsystem 128, and a visualization subsystem 132. In various implementations, the platform 100 also includes a visual and behavioral observation subsystem 196 and a user modeling subsystem 232. The various subsystems may be interconnected, such that data is transferred between the various subsystems for analysis and processing.

FIG. 1 shows the data flow throughout the platform 100 from multimodal sensor acquisition through preprocessing, foundation model embedding, vector projection into LLM space, contextual reasoning, intervention selection, and closed-loop evaluation.

### Multimodal Data Acquisition Subsystem

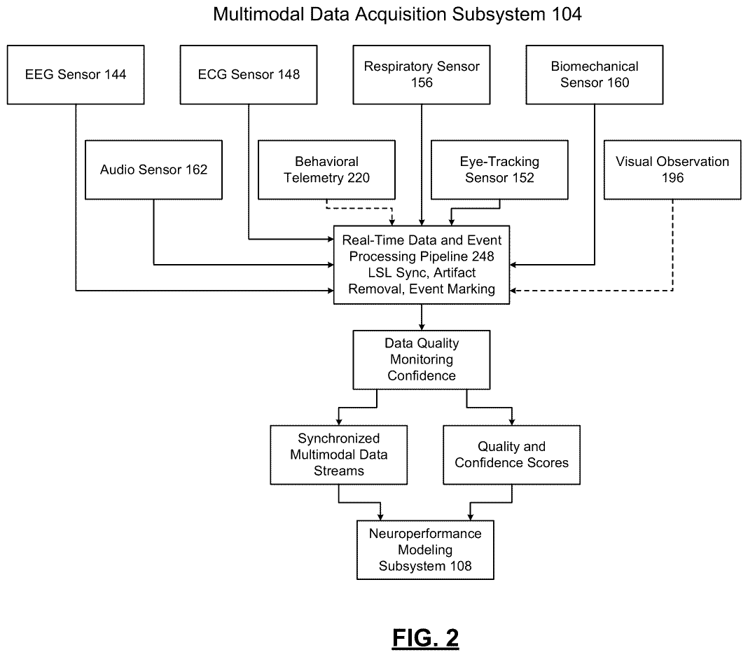

FIG. 2 illustrates the multimodal data acquisition subsystem 104. The multimodal data acquisition subsystem 104 may include specialized hardware and/or various sensors or sensor arrays to capture physiological data of the operator before, during, and/or after performance of a task. Physiological data may include any biosignals reflecting the function of biological systems, including but not limited to neural activity (EEG), cardiac activity (ECG), autonomic activity (heart rate variability), ocular activity (eye-tracking, pupil diameter), respiratory activity, muscular activity (EMG), and kinematic data (motion capture, IMU accelerometry). In various implementations, the physiological data includes at least one of: electroencephalography (EEG), electrocardiography (ECG), eye-tracking, respiratory monitoring, or biomechanical motion capture data. In various implementations, the multimodal data acquisition subsystem 104 simultaneously records EEG, ECG, eye-tracking, respiratory monitoring, and biomechanical motion capture data. The multimodal data acquisition subsystem 104 may also capture other types of physiological data and may capture such data asynchronously within the scope of the present disclosure.

### Electroencephalography

In various implementations, the multimodal data acquisition subsystem 104 includes an EEG sensor 144. In various implementations, the EEG sensor 144 is an array of sensors.

In various implementations, the EEG sensor 144 utilizes a clinical-grade amplifier with 64 electrodes positioned according to the international 10-20 system, sampling at 500 Hz with 24-bit resolution. Electrode impedance is maintained below 5 kΩ through saline-based contact media. The amplifier bandwidth spans 0.1 Hz to 200 Hz. Individual electrode signals are recorded referenced to a common reference (such as the central midline electrode (Cz) or linked earlobes) and are later re-referenced digitally to a common average reference for artifact reduction. EEG is the primary sensor for direct measurement of neural oscillations across frequency bands: delta (0.5-4 Hz), theta (4-8 Hz), alpha (8-12 Hz), beta (13-30 Hz), gamma (30-100 Hz), and high-gamma (100-200 Hz). Performance-relevant oscillations may include frontal midline theta for sustained attention, occipital alpha for visual processing, motor cortex beta for movement preparation, and parietal alpha for working memory.

While one example of the EEG sensor 144 is described above, the EEG sensor 144 may be any sensor that is capable of capturing EEG signals within the scope of the present disclosure.

### Electrocardiography

In various implementations, the multimodal data acquisition subsystem 104 includes an ECG sensor 148. In various implementations, the ECG sensor 148 is an array of sensors.

In various implementations, the ECG sensor 148 records the electrical activity of the heart via a three-lead configuration (placing electrodes on the right shoulder, left shoulder, and left hip) sampled at 1 kHz with 16-bit resolution. QRS complex detection is performed in real-time to extract inter-beat intervals (IBIs) and compute heart rate variability (HRV) metrics, including RMSSD (root mean square of successive differences), pNN50 (percentage of inter-beat intervals differing by more than 50 ms), and HRV coherence measures reflecting autonomic balance. ECG provides real-time heart rate, beat-by-beat variability reflecting parasympathetic tone and stress resilience, and coherence metrics indicating cardiovascular-respiratory coupling.

While one example of the ECG sensor 148 is described above, the ECG sensor 148 may be any sensor that is capable of capturing ECG signals within the scope of the present disclosure.

### Eye-Tracking

In various implementations, the multimodal data acquisition subsystem 104 includes an eye-tracking sensor 152. In various implementations, the eye-tracking sensor 152 is an array of sensors.

In various implementations, the eye-tracking sensor 152 is a head-mounted eye-tracker that records at least one of pupil position, pupil size (diameter), gaze direction, and fixation duration at a 120 Hz sampling rate. Gaze data may be calibrated to the visual display or real-world scene in front of the subject. Fixation may be detected when gaze velocity falls below a predetermined threshold (such as 30 degrees per second). From raw gaze data, the platform 100 may compute at least one of: quiet eye duration (time of stable gaze on target before action initiation), saccade frequency and amplitude, pupil diameter (reflecting cognitive load and arousal), or gaze entropy (measuring attention breadth). Eye-tracking may be especially important in sports applications where visual attention and gaze control directly determine performance.

In various implementations, the pupil diameter signal acquired by the eye-tracking sensor 152 is corrected for luminance-driven pupil light reflex artifacts before being used in cognitive state assessment. The correction accounts for at least the luminance of the screen content in the region of the operator's current gaze fixation, such that changes in pupil diameter attributable to changes in display brightness are separated from changes attributable to cognitive load, arousal, and attentional effort. The corrected pupil signal may be further decomposed into tonic (slowly varying baseline) and phasic (event-related transient) components. The tonic component reflects sustained arousal level, and the phasic component reflects moment-to-moment cognitive responses to specific stimuli or task events. In various implementations, the correction further accounts for gaze position effects on measured pupil diameter. The pupil correction and decomposition may be performed using any suitable signal processing methodology without departing from the scope of the disclosed embodiments.

While one example of the eye-tracking sensor 152 is described above, the eye-tracking sensor 152 may be any sensor that is capable of capturing information about the eye movement of the operator within the scope of the present disclosure.

### Respiratory Monitoring

In various implementations, the multimodal data acquisition subsystem 104 includes a respiratory monitoring sensor 156. In various implementations, the respiratory monitoring sensor 156 is an array of sensors.

The respiratory monitoring sensor 156 may include any sensing device or combination of devices that detect, measure, and capture data indicative of an operator's respiratory activity. The respiratory monitoring sensor 156 may include at least one of: a thermistor or thermocouple positioned to detect airflow temperature variations at or near the nose or mouth, a nasal pressure transducer, a piezoelectric or strain gauge-based thoracic or abdominal effort belt, an impedance pneumography sensor, a capnography sensor for measuring end-tidal carbon dioxide concentration, a pulse oximeter for detecting blood oxygen saturation levels as a proxy for respiratory function, an acoustic sensor for detecting breath sounds, or an optical or infrared-based sensor for detecting chest wall motion. The respiratory monitoring sensor 156 may be implemented as a wearable device, a bedside or tabletop device, a patch adhered to the operator's skin, or any other suitable form factor, and may communicate captured respiratory data to one or more downstream processing components via a wired or wireless communication link.

In various implementations, the respiratory monitoring sensor 156 acquires breathing rate, depth, and pattern data using a chest-mounted strain gauge or respiratory inductance plethysmography belt sampled at 25-50 Hz. From the raw respiratory waveform, the platform 100 computes: instantaneous breathing rate, inspiratory-to-expiratory ratio, respiratory variability, and respiratory-cardiac coherence (the degree of coupling between breathing phase and heart rate oscillations). Respiratory metrics provide indicators of arousal regulation, stress response, and the effectiveness of breathing-based interventions. Slow, controlled breathing (4-6 breaths per minute) is associated with parasympathetic activation and improved autonomic balance, while rapid, shallow breathing indicates sympathetic activation and elevated stress.

The respiratory data captured by the respiratory monitoring sensor 156 may include, without limitation, respiratory rate, tidal volume, inspiratory and expiratory flow rates, breath-to-breath timing intervals, duty cycle, the presence or absence of apneic or hypopneic events, and any derived or composite metrics obtainable therefrom. The respiratory monitoring sensor 156 may be any sensor or sensor system capable of generating signals from which respiratory parameters can be extracted or inferred without departing from the scope of the disclosed embodiments.

### Biomechanical and Motion Capture

In various implementations, the multimodal data acquisition subsystem 104 includes a biomechanical and motion capture sensor 160. In various implementations, the biomechanical and motion capture sensor 160 is an array of sensors.

The biomechanical and motion capture sensor 160 may include any sensing device or combination of devices that detect, measure, and capture data indicative of at least one of: the operator's physical movement, body position, joint articulation, or overall biomechanical activity. The biomechanical and motion capture sensor 160 may include at least one of: an inertial measurement unit (IMU) including accelerometers, gyroscopes, and/or magnetometers, an optical motion capture system employing a set of cameras and reflective or active markers placed on the operator's body, a marker-less vision-based motion capture system using depth cameras or stereoscopic imaging, an electro-goniometer for measuring joint angles, a force plate or pressure-sensing array for capturing ground reaction forces and center of pressure data, an electromyography (EMG) sensor for detecting skeletal muscle activation patterns, a flex sensor or bend sensor positioned along a limb or joint, or a magnetic tracking system employing electromagnetic field generators and receivers affixed to body segments. The biomechanical and motion capture sensor 160 may be implemented as at least one of: a wearable device affixed to one or more body segments, an external camera-based system, a sensor-embedded garment or suit, an instrumented insole or footwear, or any other suitable form factor, and may communicate captured biomechanical and motion data to one or more downstream processing components via a wired or wireless communication link.

In various implementations, the biomechanical and motion capture sensor 160 acquires body movement and posture data using inertial measurement units (IMUs) comprising tri-axial accelerometers, gyroscopes, and magnetometers, sampled at 50-200 Hz. IMUs may be embedded in wearable devices (e.g., chest straps, wristbands, headbands) or in standalone motion capture hardware. From the raw inertial data, the platform 100 computes: movement intensity and frequency, postural stability, head movement patterns, gestural activity, and a composite movement proxy reflecting overall physical engagement or restlessness. For knowledge work applications, accelerometer data from a chest-mounted sensor provides indicators of postural shifts, fidgeting, and physical engagement that complement the behavioral telemetry from keyboard and pointing device interactions. For sports applications, biomechanical data captures movement kinematics, timing, and consistency relevant to motor skill execution

The biomechanical and motion data captured by the biomechanical and motion capture sensor 160 may include at least one of: linear and angular displacement, velocity, and acceleration of one or more body segments, joint range of motion, gait parameters such as stride length, cadence, stance and swing phase durations, and step symmetry, postural sway metrics, kinetic data including forces, moments, and torques acting on or generated by the body, muscle activation timing and amplitude, or any derived or composite metrics obtainable therefrom. The biomechanical and motion capture sensor 160 may be any sensor or sensor system capable of generating signals from which biomechanical or motion parameters can be extracted or inferred without departing from the scope of the disclosed embodiments.

### Audio and Speech Capture

In various implementations, the multimodal data acquisition subsystem 104 includes an audio capture sensor 162. In various implementations, the audio capture sensor 162 is an array of sensors.

The audio capture sensor 162 may acquire at least one of speech, system sound, or ambient sound from the operator's environment. The audio capture sensor 162 may support at least one of: voice-based interaction with the platform 100, auditory attention assessment through cortical speech tracking when combined with electroencephalography data from the EEG sensor 144, speaker identification and diarization for collaboration context detection, or speech content analysis for task context enrichment. In various implementations, the audio capture sensor 162 stores raw broadband speech envelope signals as a privacy-preserving representation suitable for cortical speech tracking analysis without retaining intelligible speech content.

The audio capture sensor 162 may be any sensor capable of acquiring at least one of speech, system sound, or ambient sound from the operator's environment without departing from the scope of the present disclosure.

### Preprocessing and Artifact Removal

In various implementations, the multimodal data acquisition subsystem 104 pre-processes the raw physiological data captured by the specialized hardware and/or various sensors or sensor arrays to remove noise or artifacts from the data. In various implementations, the pre-processing is performed in real time. The pre-processing may include at least one of: (1) bandpass (high-pass and/or low-pass) filtering to remove low-frequency noise and/or high-frequency electronic noise; (2) notch filtering (for example, at 50/60 Hz) and harmonics to remove mains power interference; (3) independent component analysis (ICA) to identify and remove artifactual components (such as eye movement artifacts, muscle noise, cardiac artifacts, etc.); (4) visual artifact marking, where researchers flag obvious movement artifacts exceeding amplitude or frequency thresholds; or (5) adaptive baseline correction to account for slow shifts in signal properties over recording sessions.

### Event Marking and Timing Synchronization

In various implementations, the multimodal data acquisition subsystem 104 includes a real-time data and event processing pipeline 248 that acquires, timestamps, synchronizes, and routes signal streams produced by the EEG sensor 144, ECG sensor 148, eye-tracking sensor 152, respiratory monitoring sensor 156, biomechanical and motion capture sensor 160, or audio capture sensor 162 before they are consumed by downstream subsystems. Behavioral telemetry from the behavioral telemetry module 220 and visual observation data from the visual and behavioral observation subsystem 196 may also be ingested into the pipeline 248, such that physiological, behavioral, and visual modalities share a common timing reference and event namespace.

In various implementations, synchronization is performed using the Lab Streaming Layer (LSL) protocol or an equivalent time-aligned streaming framework, where each sensor device publishes samples into a network-accessible stream identified by a stream name and stream-source identifier, and each sample carries a high-precision timestamp aligned to a shared clock reference. In various implementations, the real-time data and event processing pipeline 248 subscribes to active streams, corrects for per-device hardware latency and clock drift, and produces a synchronized multimodal output where samples from different modalities can be aligned to within a configurable temporal tolerance (for example, within a few milliseconds).

In various implementations, event marking is performed via a dedicated event or marker stream exposed by the real-time data and event processing pipeline 248. The event stream may record, without limitation: task-initiated events (stimulus onset, trial start, trial end, task boundary); operator-initiated events (button press, response, decision point, application switch); system-initiated events (intervention delivered, intervention accepted, intervention dismissed, agent query received, agent query answered); and externally sourced events (calendar events, remote notifications, contextual transitions). In various implementations, each event is timestamped against the shared clock reference and persisted alongside the corresponding physiological, behavioral, and visual streams, enabling precise post-hoc alignment of physiological state with task context.

In various implementations, the real-time data and event processing pipeline 248 further includes a data quality monitoring module configured to continuously evaluate the integrity of each incoming stream, producing confidence scores that flag at least one of: signal dropouts, electrode-impedance elevation, signal saturation or clipping, motion artifacts, packet loss, and clock drift. In various implementations, confidence scores are emitted alongside the synchronized streams and propagated to downstream subsystems so that assessments performed on degraded data can be weighted or suppressed accordingly. The real-time data and event processing pipeline 248 may execute on the operator's local device, on an edge computing node, or on a remote server, and may communicate with sensor devices via wired connections, local wireless connections, or network-based streaming protocols.

In various implementations, the multimodal data acquisition subsystem 104 may mark portions of the data that align with a specific event that occurred while the operator was performing the task. In various implementations, the multimodal data acquisition subsystem 104 may perform time synchronization on the raw data to align the different data streams gathered by the multimodal data acquisition subsystem 104.

### Neuroperformance Modeling Subsystem

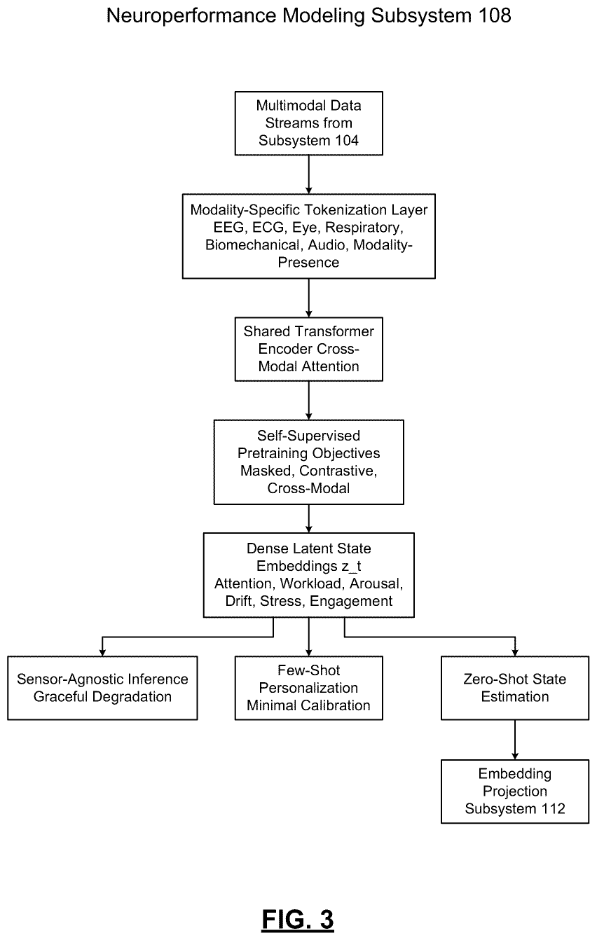

FIG. 3 illustrates the neuroperformance modeling subsystem 108. In various implementations, the neuroperformance modeling subsystem 108 includes a first model. In various implementations, the first model is a neuroperformance foundation model. The neuroperformance foundation model may be a large self-supervised learning artificial intelligence model architecture that maps raw multimodal physiological data (for example, from the multimodal data acquisition subsystem 104) to generalizable latent state embeddings. As used herein, embedding or latent embedding may refer to a continuous-valued vector representation of data in a learned feature space, typically produced by neural networks, where semantic and structural relationships are preserved (similar data points have similar embeddings).

The neuroperformance foundation model may produce embeddings from the physiological data that: (1) capture generalizable aspects of physiological organization; (2) are robust to individual differences in absolute signal magnitudes; (3) transfer well to new task domains and individuals through fine-tuning; and (4) encode sufficient information to support downstream prediction of performance outcomes and cognitive states.

The neuroperformance foundation model may use a transformer-based architecture operating on multimodal time-series data. Input signals (for example, data from the multimodal data acquisition subsystem 104) are segmented into overlapping temporal windows (typically, 2-5 seconds) and encoded into feature vectors for each modality. These multimodal features are concatenated and fed into a multi-head self-attention transformer that learns dependencies within and across physiological channels. The transformer produces latent embeddings of fixed dimension (typically, 768-1024 dimensions) representing the integrated physiological state during each temporal window.

The neuroperformance foundation model may be trained on diverse datasets spanning multiple task domains, individuals, and conditions, enabling it to learn domain-general principles of physiological organization. In various implementations, the neuroperformance foundation model is trained using multiple self-supervised learning objectives: (1) temporal contrastive learning, where embeddings of temporally adjacent windows are pulled together while embeddings of distant windows are pushed apart, teaching the model temporal continuity of physiological state; (2) multimodal contrastive learning, where embeddings derived from different sensor combinations are contrasted, teaching the model robust representation of state independent of sensor configuration; (3) masked signal reconstruction, where portions of input signals are masked and the model predicts the masked values from remaining channels, teaching cross-modal dependencies; and (4) temporal prediction, where the model predicts future physiological state from present embeddings, teaching causal temporal structure.

These self-supervised objectives are combined through weighted multi-task learning. Pretraining occurs on large public datasets (such as PhysioNet, EEGEyeNet, sports performance datasets) and on de-identified proprietary performance data, as well as on ecological data collected during knowledge work tasks, including software development, document authoring, data analysis, and research activities.

In various implementations, the neuroperformance foundation model is task-agnostic during pretraining, learning only from the structure of physiological signals without task-specific labels. This enables zero-shot transfer to entirely new domains. Fine-tuning on domain-specific labeled data (aviation, athletics, trading, knowledge work, etc.) adapts the embeddings to task-relevant features while preserving learned representations of fundamental physiological organization.

### Embedding Projection Subsystem

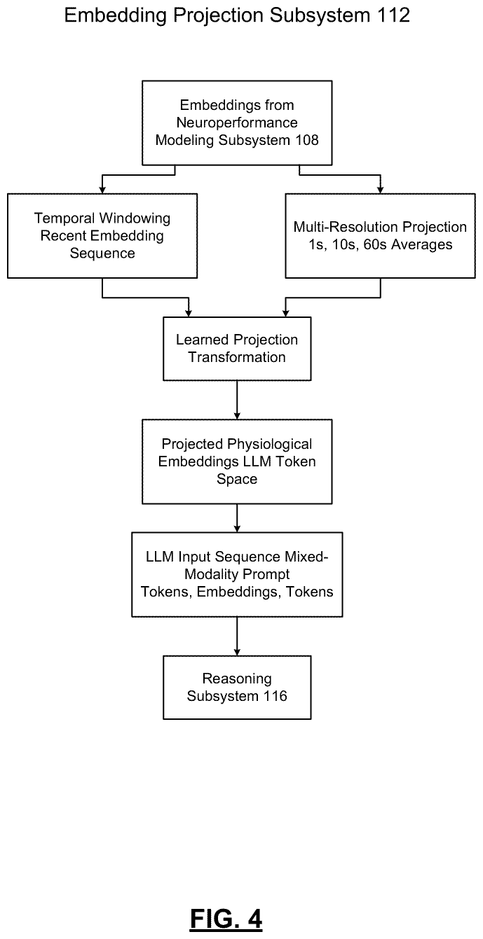

Large language models (LLMs) operate in a discrete token space, making it impossible to directly process continuous-valued physiological embeddings. The embedding projection subsystem 112, which is illustrated in FIG. 4, addresses this problem by using Vector-ICL (In-Context Learning) to map (e.g., convert) physiological state embeddings (for example, the embeddings produced by the neuroperformance foundation model) into the native token embedding space of the LLM, enabling the LLM to perform native reasoning over physiological data. As used herein, a large language model may refer to transformer-based neural network models trained on vast corpora of text data, including but not limited to GPT-4, Claude, Gemini, and other models with contextual reasoning and natural language generation capabilities.

In various implementations, the embedding projection subsystem 112 is a learned linear transformation or shallow neural network that maps 768-dimensional physiological embeddings from the neuroperformance foundation model to the embedding space of the target LLM (for example, a 4096-dimensional space for a large LLM like GPT-4). This transformation may be trained through supervised learning. For example, a labeled dataset of (physiological embeddings, desired LLM outputs) may be used to learn the mapping that produces embeddings whose token-space neighbors correspond to tokens describing the physiological state.

Once projected into LLM token space, physiological embeddings behave like continuous token embeddings. The LLM's attention mechanism can attend to physiological embeddings just as it attends to discretized tokens, enabling seamless multimodal reasoning. Physiological embeddings can be interspersed with discrete tokens in the input sequence, creating a mixed-modality prompt, such as: “[TOKENS: operator name, task, timestamp] [PHYSIO: embedded state] [TOKENS: task description] [PHYSIO: embedded state from 2 seconds later]”, enabling the LLM to reason over time-aligned physiological and contextual information.

The embedding projection subsystem 112 may enable few-shot learning in the physiological domain. By providing a few examples of (physiological state, performance outcome) pairs in the prompt, the LLM can learn the pattern and apply it to new states, thereby performing in-context learning of physiological state interpretation, which is more data-efficient than training separate supervised models.

### Reasoning Subsystem

The reasoning subsystem 116 may receive inputs, analyze the inputs, and generate outputs based on the inputs. In various implementations, the reasoning subsystem includes a second model. In various implementations, the second model is a context-aware LLM reasoning engine. The LLM reasoning engine may accept as inputs at least one of: (1) real-time physiological embeddings from the neuroperformance modeling subsystem 108; (2) task and performance context related to the task being performed by the operator (task description, stimulus information, recent performance outcomes); (3) a structured prompt describing the reasoning task; or (4) few-shot examples. The LLM reasoning engine may generate outputs including at least one of: real-time state assessments, performance predictions, causal reasoning about performance degradation, or intervention recommendations related to the task being performed.

In various implementations, the LLM reasoning engine is fed the structured prompt using a multi-stage approach. One example of the multi-stage prompting approach is described more below. Stage 1 prompt generates a high-level state assessment: “Based on the physiological signals shown, what is the current state of the operator (alert, fatigued, stressed, focused, distracted, in flow state, etc.)?” Stage 2 prompts generate detailed analysis of specific dimensions: “What is the operator's current level of attention focus (1-10 scale) and how confident are you in this estimate?” Stage 3 prompts generate predictions: “Based on this physiological state, what is the probability the operator will make an error in the next 30 seconds?” Stage 4 prompts generate recommendations: “What is the single highest-impact intervention that could improve this operator's performance right now?”

In various implementations, the prompt provided to the LLM reasoning engine includes domain-specific context to ground its reasoning. For example, for an aviation application (where the operator is a pilot and the task is flying a plane), the prompt includes: “You are an expert aviation psychologist analyzing pilot performance. The pilot is currently at cruise altitude, 4 hours into an 8-hour flight.” For an athletics application, the prompt includes: “You are an elite sports psychology coach analyzing an athlete's pre-performance state. The athlete is about to attempt a basketball free throw, their 48th attempt in this session.”

In various implementations, chain-of-thought prompting is used to encourage detailed reasoning by the LLM reasoning engine: “First, identify the dominant frequency bands and their laterality. Second, interpret the autonomic balance. Third, assess attention allocation from eye-tracking data. Fourth, synthesize these into an integrated state assessment. Finally, recommend an intervention.”

In various implementations, the LLM reasoning engine also supports interactive natural language queries enabling coaches, performers, and medical professionals to ask questions about performance, such as “Why did my performance drop between attempts 25 and 30?”; “What breathing pattern would optimize my state right now?”; or “Which sessions show the best flow state indicators?” In response, the LLM reasoning engine retrieves relevant physiological data, generates contextual analysis, and produces detailed natural language responses integrating domain expertise with data-driven insights.

In various implementations, the task context information received by the reasoning subsystem 116 is derived from the visual and behavioral observation subsystem 196, including task classifications, error state assessments, content complexity ratings, and gaze-content alignment determinations produced by the hierarchical multimodal machine learning pipeline 212. In various implementations, a physio-adaptive interaction policy generated by the reasoning subsystem 116 may be exposed through a structured interface protocol to external software agents, enabling any connected software agent to adjust its behavior based on the operator's current cognitive state.

### Intervention Subsystem

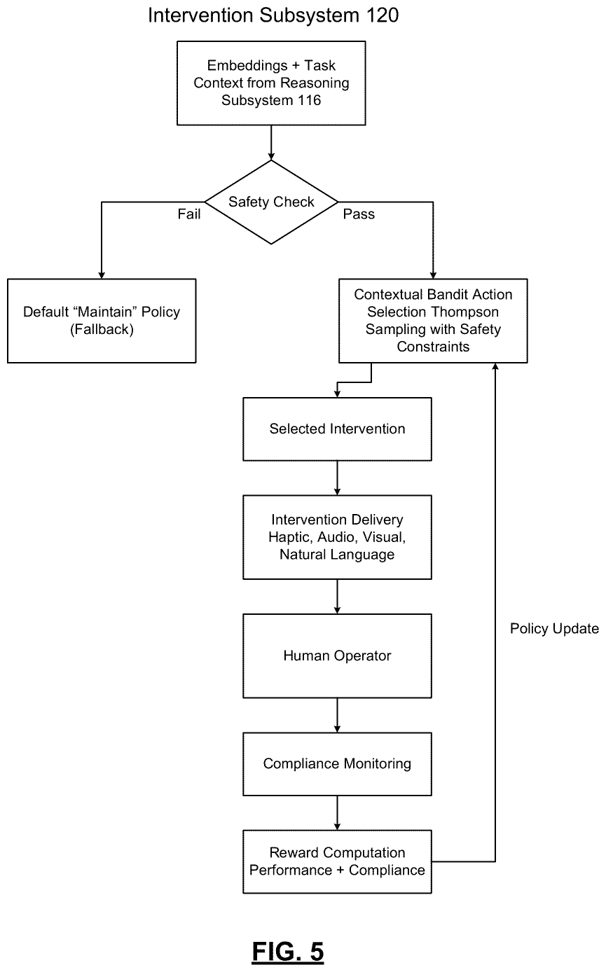

FIG. 5 illustrates the intervention subsystem 120. In various implementations, the intervention subsystem 120 is a closed-loop intervention subsystem. In various implementations, the intervention subsystem 120 selects and delivers time-optimal interventions (for example, feedback) in real-time. Rather than reactive feedback triggered when a problem is detected, the intervention subsystem 120 is proactive, predicting performance degradation and intervening preventatively.

In various implementations, the intervention subsystem recommends interventions according to an intervention selection policy. The intervention selection policy may be trained using hierarchical reinforcement learning. In various implementations, the intervention selection policy has a two-tier structure, including a high-level policy and a low-level policy. The high-level policy selects an intervention class from a set of intervention classes based on the estimated state and recent performance trends. In various implementations, the set of intervention classes includes a breathing exercise class, an attention refocusing class, a motor preparation cue class, an encouragement class, a minimal intervention class, an error-fix with contextual explanation class, a comprehension-explain class for specific content, a break-suggest class based on fatigue detection, a flow-protect class via interruption suppression, a strategy-hint class for stuck states, and a daily performance summary class with actionable recommendations. The low-level policy selects specific parameters for the chosen intervention class. For example, if the breathing exercise is selected, the low-level policy determines the specific breathing rhythm to recommend based on the individual's neurotype.

In various implementations, the selected intervention explicitly accounts for compliance: individuals vary in their responsiveness to interventions. The intervention subsystem 120 may model an individual's intervention response pattern, such as through Bayesian inference. A compliance probability for each intervention type is estimated from historical response data. When selecting interventions, the intervention subsystem 120 optimizes for expected improvement in performance, accounting for both the intervention's effect size and the individual's compliance probability. As a result, low-compliance-probability interventions are deprioritized, even if they would be highly effective if followed.

As discussed more below, interventions are delivered through multiple modalities. The modalities may include at least one of: (1) haptic feedback via wearable vibration motors (immediate, non-intrusive) worn by the operator; (2) audio cues (tone, spoken instructions); (3) visual indicators displayed on the operator's equipment or on a coach's tablet; or (4) natural language coaching messages. In various implementations, the modality is selected based on at least one of: the task context or individual preference. For example, for a free throw shooter, haptic feedback on the wrist is non-disruptive and provides a reminder to check breathing, while for a pilot, an audio alert combined with natural language explanation may be more appropriate.

### Multi-Timescale Feedback Delivery

In various implementations, the intervention subsystem 120 provides feedback on multiple time scales, including immediate feedback, short-term feedback, session-level feedback, and long-term feedback.

Immediate feedback may include real-time cues delivered to the operator during their performance of the task. Examples of immediate feedback include haptic vibration on the wrist as a breathing reminder, an audio tone indicating attention drift, and a visual indicator on an augmented reality display showing current heart rate variability coherence. In various implementations, the immediate feedback requires minimal cognitive load from the operator to understand and integrate, to avoid disrupting performance. Immediate feedback may be delivered within 500 milliseconds of the operator performing the task.

In implementations where the operator is repeatedly performing the same task (for example, a practice session of a basketball player shooting free throws), after a series of attempts (e.g., 5-10 attempts), the intervention subsystem 120 provides short-term feedback on performance trends and physiological patterns. An example of short-term feedback provided to a basketball player practicing their shooting may include: “Your accuracy has dropped 12% in the last 10 shots. Your heart rate variability shows increased stress. Try the 4-7-8 breathing rhythm before your next attempt.” This is delivered as natural language coaching generated by the LLM, possibly accompanied by trend visualizations showing performance trajectory.

In various implementations, the intervention subsystem 120 provides session-level feedback at the end of a session. In this context, a “session” may mean a period of time where the operator is repeatedly performing the same task. In various implementations, session-level feedback is based on between 30 and 120 minutes of gathered physiological data. In various implementations, session-level feedback includes comprehensive analysis of the entire session, including at least one of: performance evolution, dominant physiological states, state-to-outcome correlations, or recommendations for future training. An example of session-level feedback provided to a basketball player after a shooting session may include: “In this session, you showed your best performance (92% accuracy) when in a flow state characterized by alpha coherence >0.8 and heart rate variability RMSSD between 40-60 ms. You showed degraded performance (78% accuracy) during periods of elevated frontal theta, indicating high cognitive load. Recommendation: focus training on maintaining alpha-dominant states through breath work practice.”

In various implementations, the intervention subsystem 120 provides long-term feedback and adaptation across multiple sessions. Long-term feedback and adaptation may occur on a timescale of days to weeks. In various implementations, the intervention subsystem adapts the overall training program and individual neurotype profile of the operator based on longitudinal patterns. Examples of long-term feedback and adaptation include at least one of: identifying the operator's optimal training time of day based on circadian physiological patterns, personalizing breathing protocols based on the operator's neurotype-specific responsiveness, adjusting pre-competition meal timing to optimize physiological state, predicting upcoming periods of fatigue based on longitudinal stress markers, or recommending recovery interventions.

### Contextual Bandit and Reinforcement Learning Policy

In various implementations, the intervention selection policy is formulated as a contextual multi-armed bandit problem where the “context” is the current physiological state (extracted from the latest embedding), and each “arm” is an intervention option. The bandit algorithm learns which interventions are most effective for different states through reinforcement learning. Reward signals come from multiple sources, including at least one of: immediate performance outcome (did the intervention lead to an error or success on the next trial?), performer compliance (did the individual follow the recommendation?), or physiological response (did the intervention produce the expected state change?).

In various implementations, the intervention subsystem 120 uses Thompson sampling (a probabilistic bandit algorithm) where each intervention's efficacy is modeled as a Bayesian distribution. Interventions with higher posterior probability of high efficacy are sampled more frequently, while lower-probability interventions are occasionally sampled to explore their potential, thereby balancing exploitation (using interventions known to work) with exploration (discovering new effective interventions).

In various implementations, the intervention subsystem 120 enforces a set of safety constraints to ensure that certain interventions are never recommended in certain states (e.g., a distraction-based intervention is never recommended if the performer is already in a flow state). In various implementations, intervention frequency is limited to prevent habituation and ensure each intervention has time to take effect before the next is delivered.

### Trajectory-Aware Policy Optimization

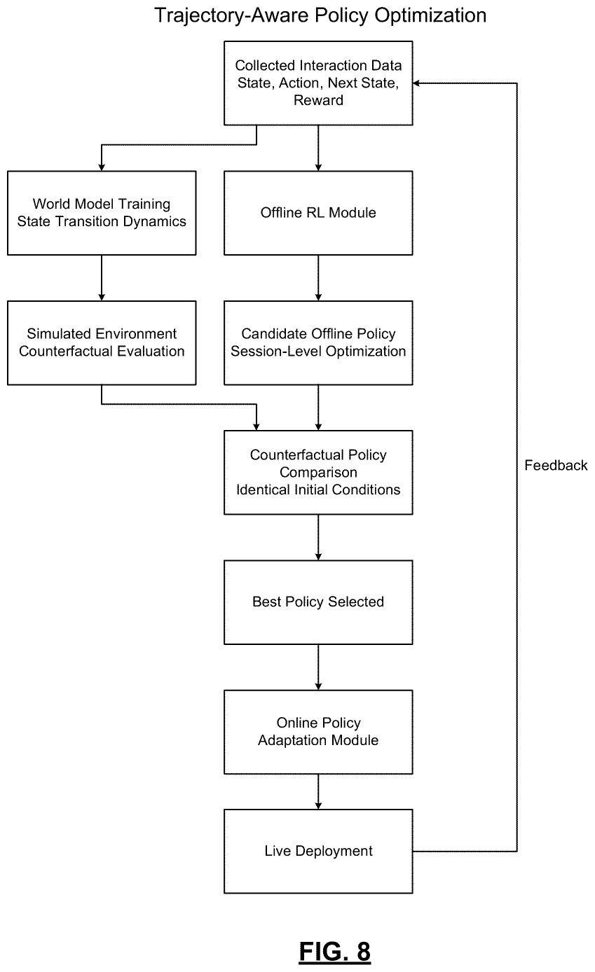

As shown in FIG. 8, in various implementations, the intervention selection policy is optimized using trajectory-aware optimization, which accounts for how current interventions reshape future physiological state and downstream performance, unlike myopic policies that optimize immediate next-step outcomes. For example, a breathing exercise delivered now will shift autonomic state, which will influence subsequent states and performance opportunities over the next several minutes.

In various implementations, the trajectory-aware optimization uses model-predictive control (MPC) to predict long-term performance outcomes using a trained model. Given the current physiological state of the operator performing the task, the platform 100 simulates multiple intervention sequences forward in time (typically, 5-30 minutes ahead), predicting how the operator's physiological state will evolve under each sequence. The intervention with the trajectory producing the best long-horizon performance outcome is selected as the intervention to be sent to the operator. This is computationally more expensive than myopic selection but produces better outcomes because it avoids short-term interventions that disrupt long-term performance trajectories.

The trained model used in the trajectory-aware optimization may be trained on historical data. Given a physiological state S_t and an intervention I_t, the model predicts S_{t+1}. In various implementations, the model is trained as a supervised neural network on thousands of (state, intervention, next state) triplets. The model captures at least one of: habituation effects (repeated identical interventions are less effective), state-dependent response (an intervention effective in one state may be ineffective in another), or temporal dynamics (some state changes take minutes to manifest).

Trajectory optimization is particularly important in sports where there are natural breaks in activity (time between free throws, time between tennis serves, time between penalty kick attempts). In various implementations, the platform 100 identifies these natural break points and uses them for intervention opportunities, selecting interventions that optimize the physiological state of the operator when the next performance attempt begins.

### Evaluation Subsystem

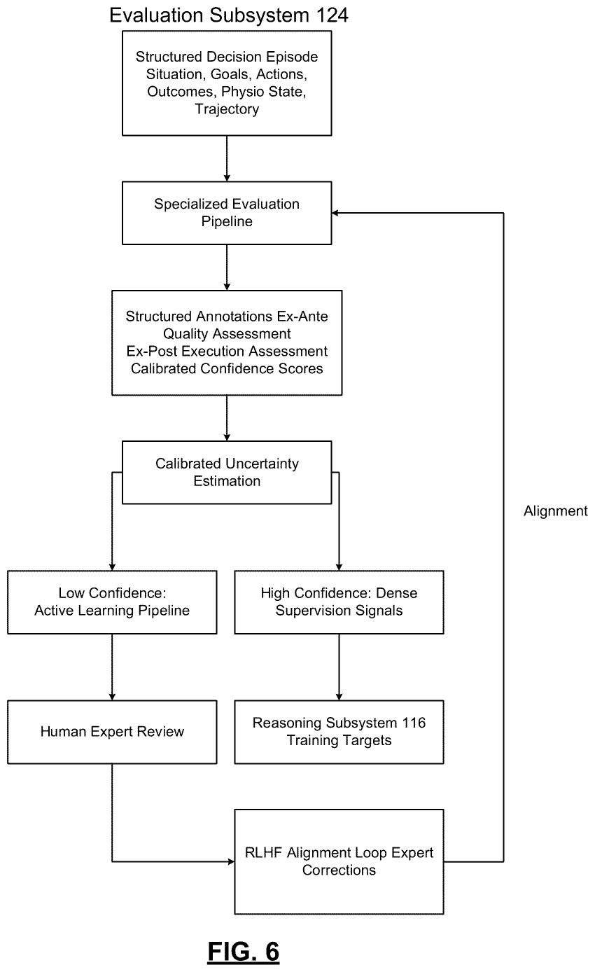

FIG. 6 illustrates the evaluation subsystem 124, which may be referred to herein as an AI judge evaluation subsystem. The evaluation subsystem 124 may generate dense, context-aware evaluations of decision quality, enabling scalable supervision, unlike traditional performance evaluation, which relies on sparse outcome signals (for example, did the performer succeed or fail?) that are insufficient for training effective models.

In various implementations, the evaluation subsystem 124 is an LLM-based evaluation system that assesses not just outcomes but the quality of the decision-making process given the information available at the time of the decision. For example, in an aviation scenario where a pilot makes a navigation decision with imperfect weather information, the evaluation subsystem 124 evaluates: “Given the weather radar data and pilot reports available at decision time, this was a reasonable decision. Outcome: successful. Decision quality: high.” If the decision led to an accident, but the decision was optimal given available information, the evaluation subsystem 124 will still rate the decision quality as high and the outcome as poor, which is a distinction critical for learning.

In various implementations, the evaluation subsystem 124 evaluates decisions on multiple dimensions. The dimensions may include at least one of: (1) information utilization (did the operator use relevant information available?); (2) reasoning quality (was the logic sound given their goal?); (3) timeliness (was the decision made at the right moment?); (4) risk awareness (was appropriate caution applied?); or (5) outcome quality (what was the objective result?). In various implementations, the evaluation subsystem 124 provides evaluations as scores (0-100 per dimension) and natural language justifications.

In various implementations, evaluations from the evaluation subsystem 124 are used as training targets for performance prediction models, such as the LLM reasoning engine of the reasoning subsystem 116. Rather than training to predict binary success/failure, the evaluations generated by the evaluation subsystem 124 may be used to train the LLM reasoning engine to predict multidimensional decision quality scores, thereby enabling the reasoning subsystem 116 to identify performance degradation earlier (decision quality drops before outcome fails) and to generate more nuanced coaching advice.

In various implementations, the evaluation subsystem 124 is calibrated on expert human evaluations. Domain experts (experienced pilots, elite coaches, trading professionals) provide quality ratings for a dataset of decision scenarios, and the evaluation subsystem 124 is fine-tuned on these expert ratings, learning the expert evaluation criteria. As a result, the evaluation subsystem 124 mimics expert decision quality assessment while scaling to evaluate millions of decisions.

As discussed more below, in various implementations, the evaluation subsystem 124 receives decision episodes from the visual and behavioral observation subsystem 196. When the hierarchical multimodal machine learning pipeline 212 detects a decision point from visual captures (e.g., the operator selecting between alternative approaches, committing to a course of action, or accepting or rejecting a suggestion from a connected software agent), the evaluation subsystem 124 may evaluate the quality of that decision using the same multi-dimensional framework described above. For knowledge work applications, the effort-effectiveness analysis performed by the visual and behavioral observation subsystem 196 may constitute an additional evaluation dimension: the ratio of behavioral investment to visible task progress provides a continuous measure of productive efficiency that complements assessments of information utilization, reasoning quality, timeliness, risk awareness, and outcome quality by the evaluation subsystem 124.

### Neurotyping Subsystem

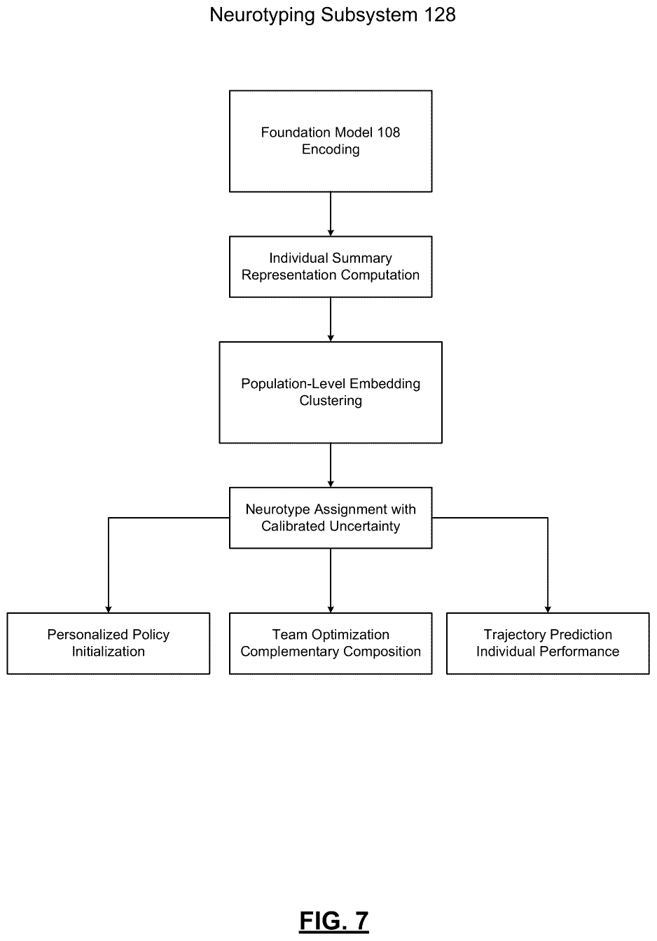

FIG. 7 illustrates the neurotyping subsystem 128. Individuals show stable, heritable patterns in their physiological response to tasks and stressors. Neurotyping characterizes these individual-specific patterns, enabling personalized optimization strategies. Rather than applying identical interventions to all individuals, the neurotyping subsystem 128 adapts the platform 100 to each operator's neurotype.

In various implementations, the neurotyping subsystem 128 extracts features from the physiological embeddings generated by the neuroperformance modeling subsystem 108 across multiple sessions. The physiological embeddings may include at least one of: baseline EEG frequency profiles, characteristic heart rate variability patterns, eye-tracking behavior style, typical response latency to stimuli, or typical amplitude of physiological responses to stressors. These features may be combined by the neurotyping subsystem 128 through unsupervised clustering to identify archetype categories.

The neurotyping subsystem 128 may recommend circadian-based training time optimization for each neurotype. Some individuals show peak neurophysiological state in morning hours (high alpha, good HRV coherence, optimal arousal), while others peak in the afternoon or evening. Training may be scheduled accordingly to maximize learning.

The neurotyping subsystem 128 may personalize the feedback from the intervention subsystem 120 based on the operator's neurotype. For example, an operator with the ‘Analytical Processor’ neurotype benefits from feedback reducing task-related theta and increasing alpha stability, whereas an ‘Intuitive Performer’ neurotype benefits from feedback enhancing beta coherence and motor cortex engagement, and a ‘Pressure Responder’ neurotype benefits from parasympathetic activation feedback (heart rate variability coherence training, slow breathing practice).

### Visual and Behavioral Observation Subsystem

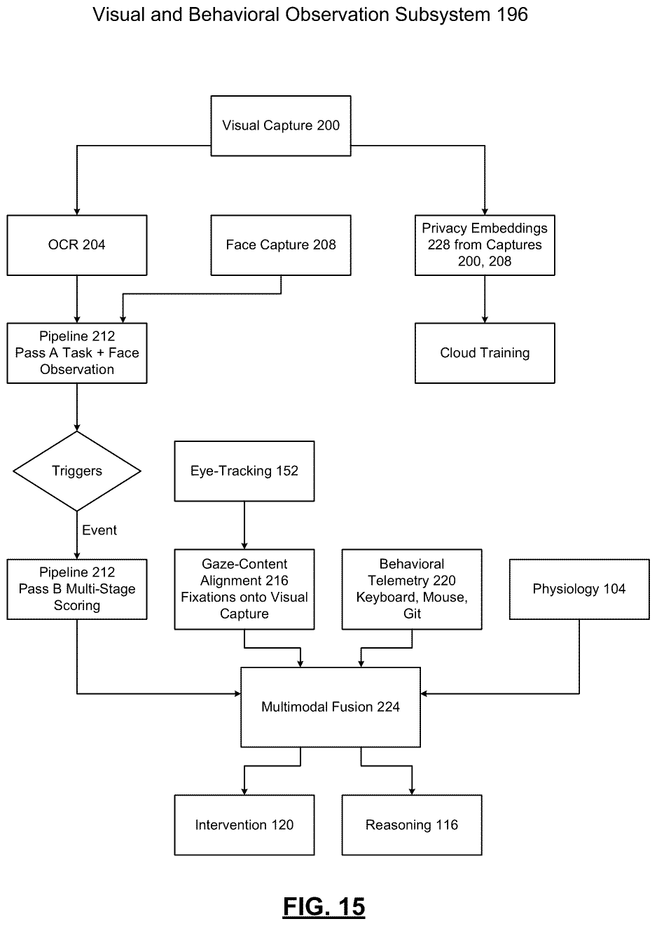

FIG. 15 illustrates a visual and behavioral observation subsystem 196 that may capture the operator's task environment through visual captures and behavioral telemetry and produces contextually-grounded cognitive state assessments. The visual and behavioral observation subsystem 196 may operate in concert with the multimodal data acquisition subsystem 104 to provide task context for performance assessment.

In various implementations, the visual and behavioral observation subsystem 196 includes a visual capture module 200 that acquires visual captures of the operator's task environment at a configurable rate (for example, 1 Hz background capture with event-triggered captures upon detected changes). Visual captures may include, without limitation: periodic screenshots of a computing display, continuous video streams, augmented reality or virtual reality viewport captures, head-mounted display feeds, camera feeds from mobile devices or wearable cameras, vehicle instrument panel captures, or any other visual representation of the environment in which the operator is performing a task. The visual capture module 200 may acquire visual captures periodically at a configurable rate, continuously as a video stream, upon detection of triggering events, or in any combination thereof.

In various implementations, the visual and behavioral observation subsystem 196 includes an optical character recognition (OCR) module 204 that extracts text content from the visual captures. In various implementations, the visual and behavioral observation subsystem 196 includes a webcam capture module 208 that periodically captures facial images of the operator for postural and fatigue assessment.

In various implementations, the visual and behavioral observation subsystem 196 includes a hierarchical multimodal machine learning pipeline 212 that processes visual captures of the operator's task environment and facial images using a multimodal machine learning model. In various implementations, the multimodal machine learning model is a vision-language model (VLM). A first observation pass employs a computationally efficient model instance operating in parallel on each captured frame to classify the operator's current task category, action type, application context, visible error states, content complexity, and progress indicators. A separate parallel face observation pass receives a cropped facial image and applies a Facial Action Coding System (FACS)-inspired rubric based on action unit analysis to assess fatigue indicators and postural signals. A set of deterministic trigger conditions, including error persistence exceeding a configurable threshold, content change detection, gaze-triggered events, keyboard or pointing device activity patterns, and temporal scheduling, gate the invocation of a deeper analysis pass. The deterministic triggers ensure that expensive model inference is invoked only when cognitively significant events are detected, resulting in computational cost that scales with cognitive events rather than wall-clock time. The deeper analysis pass employs a more capable model in a two-stage architecture. A first analysis stage receives the observation outputs, captured images, episode context comprising summaries of recent preceding situations, and a summary of physiological and behavioral indicators, and produces rubric-based evaluation evidence with qualitative reasoning for each assessment dimension. A second scoring stage receives only the first stage's textual analysis without visual captures and computes performance scores from the evidence, thereby preventing visual anchoring bias. The deeper analysis pass implements the AI Judge evaluation pattern described for the evaluation subsystem 124: rubric-based evaluation evidence is produced first, followed by deterministic scoring from the evidence.

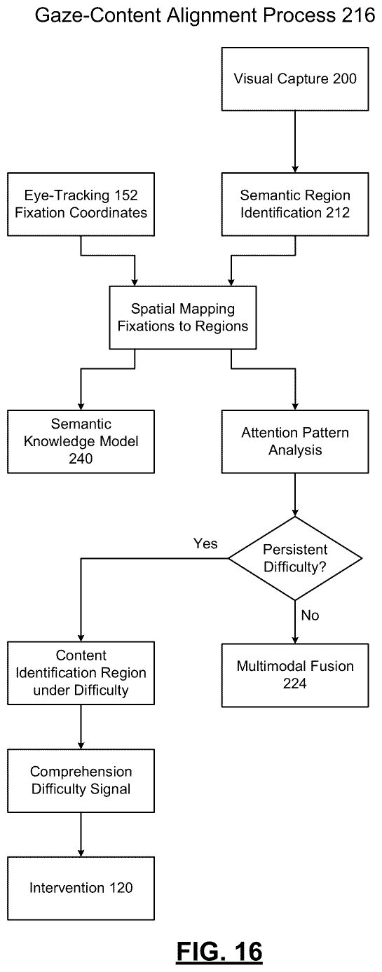

As shown in FIG. 16, a gaze-content alignment module 216 of the visual and behavioral observation subsystem 196 maps gaze fixation coordinates from the eye-tracking sensor 152 onto regions of the visual capture and determines whether the operator's visual attention is directed at task-relevant content elements. The hierarchical multimodal machine learning pipeline 212 identifies semantic regions of the visual capture (e.g., error messages, code blocks, documentation sections, UI controls, terminal output), and the gaze-content alignment module 216 computes a spatial correspondence between the operator's fixation cluster coordinates and these identified regions. When persistent gaze regressions, characterized by repeated backward saccades to previously fixated regions, are detected on a specific region of the visual capture for a configurable duration (e.g., exceeding 30 seconds), the platform 100 identifies this as a comprehension difficulty signal and may trigger a contextual intervention specific to the content of that region.

In various implementations, the visual and behavioral observation subsystem 196 includes a behavioral telemetry module 220 that acquires continuous streams of operator interaction data, including: keyboard interaction patterns (keys per minute, backspace ratio, pause rate, inter-keystroke interval variability, burst length, undo rate); pointing device dynamics (speed, path efficiency, click rate, idle ratio, scroll patterns); and version control activity (commit frequency, branch changes, merge events). These behavioral signals are synchronized with the physiological and visual observation channels via a timing synchronization infrastructure and are computed as rolling statistical summaries over sliding temporal windows (e.g., 30-second windows emitted at 1 Hz).

In various implementations, the visual and behavioral observation subsystem 196 includes a multimodal fusion module 224 that combines model-derived task assessments with physiological signals from the multimodal data acquisition subsystem 104, behavioral telemetry from the behavioral telemetry module 220, and gaze-content alignment data from the gaze-content alignment module 216 to produce composite cognitive state determinations. The multimodal fusion module 224 supports configurable weighting and inclusion of each modality at each processing stage, enabling the platform 100 to operate with any available subset of modalities while producing the most informative assessment possible given the available data.

In various implementations, the visual and behavioral observation subsystem 196 includes a privacy-preserving embedding extraction module 228 that generates semantically rich but non-reconstructable representations of visual data for model training. Visual capture embeddings are computed on-device using an encoder (e.g., a vision-language model such as CLIP or SigLIP) to produce fixed-dimensional vectors. Facial representations are computed using a geometric landmark model (e.g., MediaPipe face mesh) rather than pixel-level representations. The resulting embedding vectors and facial representations are suitable for downstream model training but cannot be used to reconstruct the original images or identify the operator.

While one example of the visual and behavioral observation subsystem 196 is described above in the context of a computing display, the visual and behavioral observation subsystem 196 may capture any visual representation of any task environment and may process such visual representations using any multimodal machine learning model within the scope of the present disclosure. For example, a behavioral telemetry module 220 may capture interaction data from any input device or development tool, and the gaze-content alignment module 216 may use any eye-tracking or attention-tracking technology, without departing from the scope of the disclosed embodiments.

In various implementations, the platform 100 may be deployed in a plurality of configurations, including: (a) a local-first configuration where sensing, inference, and intervention delivery execute on the operator's device and the structured interface protocol is exposed as a local service accessible to other applications on the same device or local network; (b) a cloud-hosted configuration where visual captures and behavioral data are transmitted to a remote server for processing; (c) an edge computing configuration where sensing occurs on the operator's device and inference executes on a nearby edge server; and (d) a hybrid configuration where time-sensitive inference and intervention delivery execute locally and model training, aggregation, and policy optimization execute on remote infrastructure. In the local-first configuration, raw physiological data, visual captures, and behavioral telemetry may remain exclusively on the operator's device, with only derived scores, embeddings, or anonymized aggregate metrics optionally transmitted to remote infrastructure for model improvement.

### User Modeling Subsystem

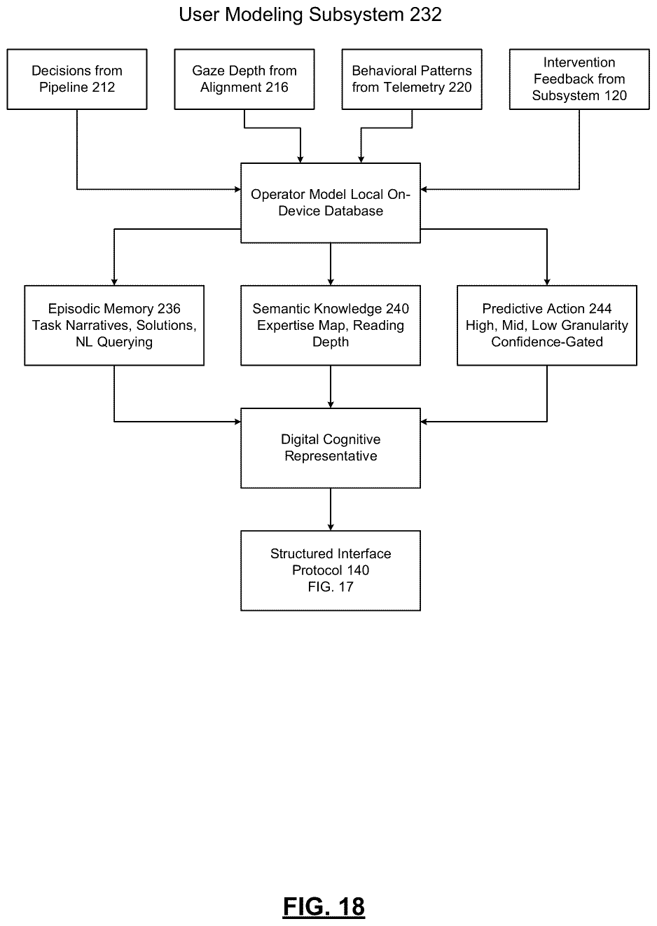

FIG. 18 illustrates the user modeling subsystem 232. In various implementations, the user modeling subsystem 232 accumulates the operator's decisions, preferences, and physiological responses over extended periods to build a comprehensive operator model that serves as the operator's digital cognitive representative. In various implementations, the user modeling subsystem 232 accumulates observations of the operator's decisions, task contexts, outcomes, behavioral patterns, cognitive preferences, and physiological responses over extended periods of use to build the comprehensive operator model. The operator model may include: a decision history database that records each detected decision point, the context in which it occurred, the action taken, the subsequent outcome, and the operator's physiological state at the time of decision; a preference model that learns the operator's decision policy from accumulated decision-outcome pairs, enabling prediction of operator preferences in novel situations; a work pattern model that characterizes the operator's temporal rhythms, including peak performance hours, optimal task-type scheduling, break patterns, and flow triggers; a vector representation store that maintains embeddings of documents, code artifacts, error messages, and other content the operator has engaged with, enabling contextual recall and continuity across sessions; and a cognitive preference model that learns the operator's preferred intervention types, timing, verbosity, and modality from intervention feedback and behavioral responses.

In various implementations, the user modeling subsystem 232 enables the platform 100 to serve as the operator's digital cognitive representative: a persistent model of the operator's cognitive characteristics, preferences, and patterns that can be queried by external software agents through a structured interface protocol. As the operator model accumulates observations over weeks and months, its predictive accuracy improves, enabling increasingly autonomous action on the operator's behalf. In various implementations, all user model data is stored on-device in a local database. The operator retains full control over the model, including the ability to inspect, modify, and delete accumulated data at any time.

In various implementations, the user modeling subsystem 232 includes an episodic memory module 236 that maintains a structured record of the operator's work episodes, including task narratives, problems encountered, solutions applied, errors resolved, and decisions made with their outcomes. Each episode is stored with its temporal context, cognitive state indicators at the time of the episode, and vector embeddings that enable semantic retrieval. The episodic memory module 236 supports natural-language querying, where the operator or a connected software agent may query the system using natural language (e.g., “when did I last work on the OAuth integration?”) and receive contextually relevant episodes retrieved via semantic similarity search over the stored embeddings.

In various implementations, the user modeling subsystem 232 includes a semantic knowledge model 240 that builds a persistent representation of the operator's domain expertise, familiarity with specific tools and technologies, and depth of engagement with different content types. The semantic knowledge model 240 is informed by the gaze-content alignment module 216: when the operator reads content, the platform 100 records not only the location of visual attention but also the depth of engagement as measured by fixation duration, regression frequency, and re-reading patterns. Over time, the semantic knowledge model 240 builds a map of the operator's knowledge landscape, distinguishing topics of deep expertise from areas of growing familiarity and domains where the operator consistently struggles.

In various implementations, the user modeling subsystem 232 supports predictive action modeling. The platform 100 observes and learns recurring action sequences, including: application switching patterns, text input patterns, navigation patterns, communication patterns, and workflow patterns. Action predictions may be provided at multiple granularity levels: high-level (predicted next task type or application), mid-level (predicted next operation within the current application), and low-level (predicted next input sequence or navigation target). Each prediction is accompanied by a confidence score. Low-confidence predictions are suppressed; high-confidence predictions may be offered as suggestions or, with the operator's consent, executed proactively.

### Visualization Subsystem

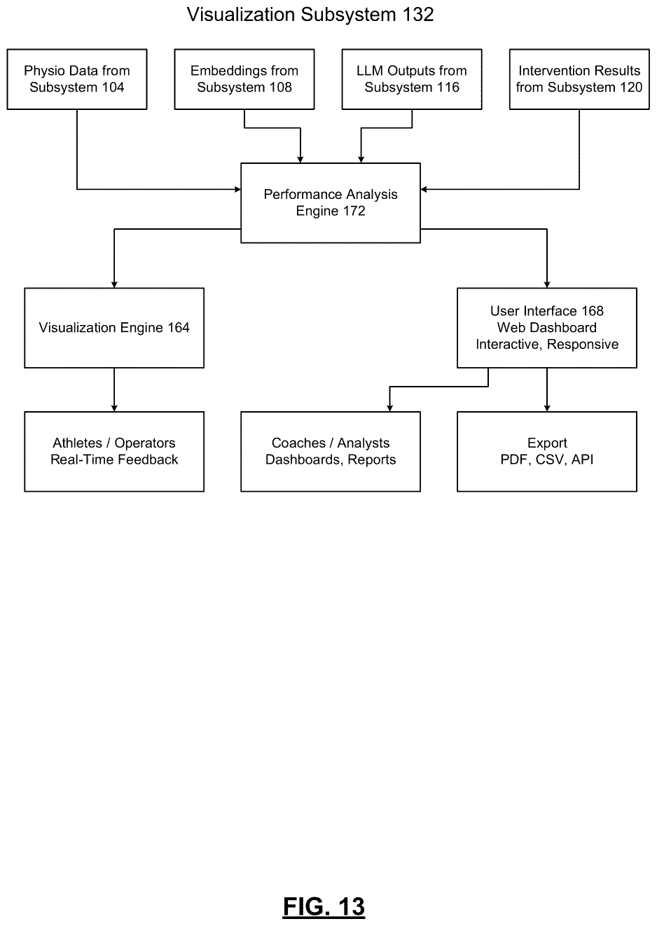

FIG. 13 illustrates the visualization subsystem 132, which provides real-time and historical analysis of performance and physiological data to the operator. The visualization subsystem 132 may include a visualization engine 164, a user interface 168, and a performance analysis engine 172.

### Visualization Engine

The visualization engine 164 may receive information from at least one of: the multimodal data acquisition subsystem 104, the neuroperformance modeling subsystem 108, the embedding projection subsystem 112, the reasoning subsystem 116, the intervention subsystem 120, the evaluation subsystem 124, or the neurotyping subsystem 128. Based on the received information, the visualization engine 164 may generate one or more plots and display the plots on the user interface 168. In various implementations, the one or more plots include at least one of: (1) time-series line plots showing raw or processed signals over time (EEG frequency bands, heart rate, breathing rate, performance metrics); (2) spectral plots showing frequency content (power spectral density for EEG); (3) heatmaps showing electrode-wise or time-frequency distributions; (4) scatter plots correlating physiological measures with performance outcomes; or (5) trend analysis showing moving averages and envelope bands. In various implementations, the visualization engine 164 updates the plots in real-time as new data arrives. In various implementations, the one or more plots are interactive, such that a user can zoom, pan, or select time windows for detailed analysis.

### Performance Analysis Engine

The performance analysis engine 172 may receive information from at least one of: the multimodal data acquisition subsystem 104, the neuroperformance modeling subsystem 108, the embedding projection subsystem 112, the reasoning subsystem 116, the intervention subsystem 120, the evaluation subsystem 124, or the neurotyping subsystem 128.

In various implementations, the performance analysis engine 172 tracks real-time performance metrics (accuracy, reaction time, response quality) and correlates physiological states with performance outcomes (e.g., performance is 18% better when the athlete is in a flow state) to identify performance bottlenecks (e.g., accuracy drops when frontal theta exceeds 10 microvolts). In various implementations, the performance analysis engine 172 generates optimization recommendations to the operator performing the task based on the identified performance bottlenecks. An example of an optimization recommendation includes: “50% of your errors occur when heart rate variability RMSSD drops below 40 ms; focus on breathing practice to increase HRV stability.” In various implementations, the optimization recommendation is displayed on the user interface 168.

### User Interface

The user interface 168 may receive and display the plots from the visualization engine 164 and the optimization recommendations from the performance analysis engine 172. The user interface 168 may include any suitable interface or combination of interface components configured to present, display, and communicate the plots from the visualization engine 164 and the optimization recommendations from the performance analysis engine 172 to a user. In various implementations, the user interface 168 is customizable, such that different users (performers, coaches, researchers, and medical professionals) see different views optimized for their role. For example, a basketball player in a performance session may see recent accuracy trend, current state (focused/stressed/fatigued), and recommended adjustments. Meanwhile, a coach for the basketball player may see team performance trends, individual athlete comparisons, and biomarker library recommendations, and a researcher may see raw data, statistical summaries, and model outputs.

In various implementations, the user interface 168 may include at least one of: a graphical user interface (GUI) rendered on a display screen, a web-based interface accessible via a browser application, a native software application interface, a heads-up display (HUD) or augmented reality (AR) overlay presented through smart glasses or a head-mounted display, a virtual reality (VR) environment, a voice-driven or conversational interface that conveys information through synthesized speech or auditory cues, a dashboard interface presenting charts, graphs, tables, gauges, indicators, or other visual representations, or an ambient or peripheral display such as an LED array, an e-ink panel, or a haptic feedback device that conveys information through non-visual channels. The user interface 168 may be rendered on or presented through any suitable computing device or display apparatus, including, without limitation, a desktop computer, a laptop computer, a tablet computer, a smartphone, a smartwatch or other wearable device, a dedicated kiosk or terminal, a smart television or connected display, a digital signage panel, an in-vehicle infotainment or instrument cluster display, a head-mounted display or smart glasses, a medical or industrial monitoring station, a set-top box connected to an external display, an Internet of Things (IoT) device with an integrated or tethered display, or any other device capable of rendering visual, auditory, or haptic output. The user interface 168 may be presented on a single device or distributed across multiple devices or displays and may support user interaction through any suitable input modality, including, without limitation, touch, gesture, voice command, mouse and keyboard, stylus, eye tracking, brain-computer interface, or any combination thereof.

In addition to the plots from the visualization engine 164 and the optimization recommendations from the performance analysis engine 172, the data and insights presented through the user interface 168 may include, without limitation, raw or processed data values, statistical summaries, trend analyses, predictive outputs, anomaly or threshold alerts, comparative visualizations, ranked or scored results, natural language summaries or narratives generated from underlying data, and any other derived, aggregated, or transformed informational output. The user interface 168 may present such data and insights in real-time, near real-time, or on a periodic, on-demand, or event-triggered basis, and may further allow the user to filter, sort, drill down into, annotate, export, or otherwise interact with the displayed information. The user interface 168 may be any interface or combination of interfaces rendered on any suitable device and capable of conveying data-derived insights to a user in a perceptible form without departing from the scope of the disclosed embodiments.

In various implementations, the user interface 168 is a web-based user interface that provides at least one of: (1) a Real-time Performance Dashboard showing current performance metrics, recent accuracy, heart rate, breathing rate, current cognitive state assessment, and current flow state probability; (2) Interactive Controls allowing selection of time windows, filtering of data, and selection of analysis options; (3) a Responsive Design supporting desktop, tablet, and mobile devices; or (4) Export Capabilities enabling download of data as CSV, visualization screenshots as PNG/PDF, and detailed reports as PDF documents.

### Real-Time Inference Pipeline

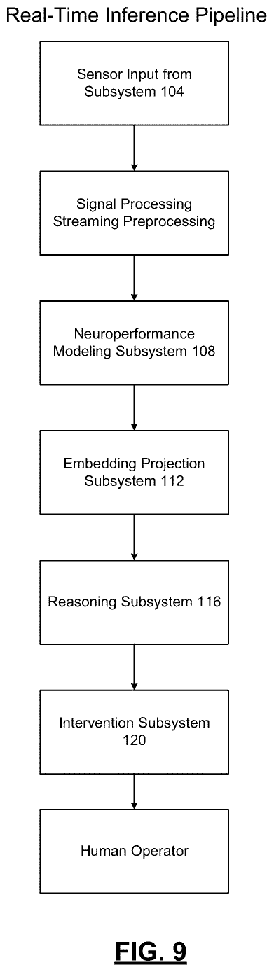

As illustrated in FIG. 9, in various implementations, the platform 100 includes a real-time inference pipeline that executes from sensor acquisition through intervention delivery in sub-second latency. As described above, raw physiological data is acquired at a high frequency (for example, 500 Hz for EEG, 1 kHz for ECG, and 200 Hz for inertial measurement units). Signals are buffered into overlapping 2-second windows. Within each window, artifact removal and preprocessing occur (ICA, filtering). The window is fed through the neuroperformance foundation model, producing a physiological embedding. The embedding is projected into LLM token space. The LLM reasoning engine generates state assessment and prediction. The intervention selection policy, given the state, selects the optimal intervention. If an intervention is warranted, it is delivered via haptic, audio, visual, or natural language channel. The outcome (performer response, subsequent performance) is logged. The total latency of the platform 100 is between 300-800 milliseconds, depending on LLM inference time.

For applications requiring lower latency (<100 ms) from the platform 100, a fast-path inference pipeline uses only the foundation model and a lightweight decision tree policy, skipping the LLM reasoning step. This trades the sophisticated reasoning of the LLM for reduced latency, useful in applications like competitive gaming or split-second military decisions where immediate reflexive feedback is critical.

For knowledge work and screen-based applications, the platform 100 may operate two complementary inference pipelines. In various implementations, the local inference pipeline executes on the operator's device: the visual capture module acquires screenshots at a configurable rate; the OCR module extracts text content; the hierarchical multimodal machine learning pipeline classifies task context, detects error states, and evaluates progress through a first processing stage; deterministic trigger conditions (error persistence, content changes, gaze events, temporal scheduling) gate invocation of a deeper analysis pass; behavioral telemetry (keyboard, pointing device, version control) is computed as rolling statistical summaries; and the multimodal fusion module combines local observations with any available physiological signals to produce cognitive state assessments and trigger interventions. In various implementations, the local pipeline is optimized for latency (sub-second from observation to intervention) and privacy (raw visual captures and behavioral data remain on-device). In various implementations, the cloud inference pipeline handles compute-intensive operations: synchronized multimodal physiological streams are processed through the neuroperformance foundation model to derive biomarkers; model training and policy optimization execute on GPU-accelerated infrastructure; and longitudinal analyses including neurotyping refinement, user model aggregation, and biomarker validation are performed on accumulated data. In various implementations, the two pipelines converge at the multimodal fusion module, which can operate locally using only locally-available signals or incorporate cloud-derived assessments and model updates when connectivity permits.

In various implementations, the platform 100 supports a dual-pipeline architecture for knowledge work and other screen-based task domains. In various implementations, a local inference pipeline executes on the operator's device and comprises: visual capture acquisition at configurable rates, OCR text extraction, behavioral telemetry collection, deterministic trigger evaluation, hierarchical multimodal machine learning model inference for task classification and error detection, deterministic stuck detection rules, and lightweight intervention delivery via system notifications. In various implementations, the local pipeline operates with sub-second latency and requires no network connectivity, preserving operator privacy by retaining raw data on-device. In various implementations, a cloud inference pipeline receives anonymized embeddings, aggregated behavioral metrics, and consented telemetry from the local pipeline and performs: multimodal signal synchronization across physiological, visual, and behavioral streams, foundation model embedding computation, biomarker derivation from synchronized signals relative to incoming cognitive events, intervention policy optimization via reinforcement learning, longitudinal user model refinement, and global model training across the population of consenting operators. In various implementations, the cloud pipeline operates at higher latency (seconds to minutes) but provides the heavy computational resources required for model training and cross-user learning. In various implementations, the two pipelines are connected by a synchronization layer that ensures local intervention decisions benefit from cloud-derived model improvements while maintaining real-time responsiveness.

### Cognitive Primitive Extraction Subsystem

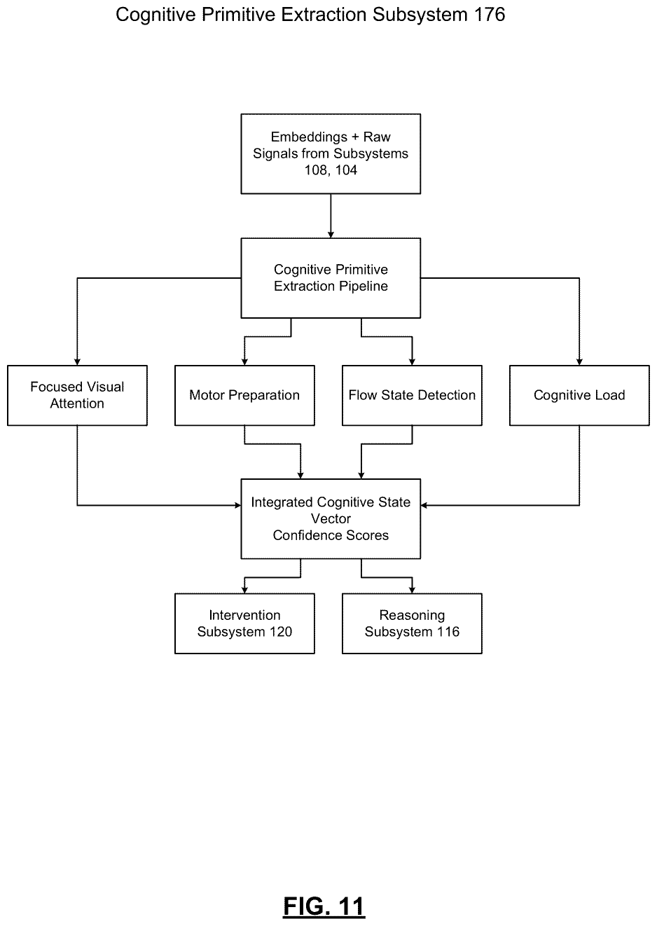

In various implementations, the platform 100 includes a cognitive primitive extraction subsystem 176 that determines at least one of: focused visual attention of the operator, motor preparation of the operator, flow state detection of the operator, or cognitive load assessment of the operator. FIG. 11 illustrates the cognitive primitive extraction subsystem 176. In various implementations, this is the base measure of the system, which is linked to known cognitive constructs. For example, three main types of physiological inputs include raw or minimally physiology, cognitive primitives derived from known signals and measures, and transformer and projector embeddings that are learned via foundation models.

### Focused Visual Attention

Focused visual attention is characterized by alpha power (8-12 Hz) modulation in occipital regions showing task-specific increases during visual attention allocation, low-frequency eye-tracking entropy indicating stable fixation rather than scanning, and pupil diameter changes reflecting attentional effort. The cognitive primitive extraction subsystem 176 may correlate these measures with performance outcomes, identifying the alpha power and fixation stability thresholds above which visual attention is classified as “focused.” In various implementations, cross-individual models are developed from dozens of athletes, then personalized to individual baselines.

### Motor Preparation

Motor preparation is indicated by beta desynchronization (13-30 Hz power decrease) in motor cortex preceding movement, movement-related cortical potentials (Bereitschaftspotential) detected from central electrodes, and electromyographic (EMG) pre-activation patterns showing muscles being readied before overt movement. The cognitive primitive extraction subsystem 176 may identify physiological signatures of when the motor system is optimally prepared for execution.

### Flow State Detection

Flow states (psychological absorption, optimal challenge, performance without conscious deliberation) are characterized by an increased theta/alpha ratio in frontal regions, indicating reduced prefrontal deliberation while maintaining arousal, reduced default mode network activity (less self-referential thought), and optimal autonomic arousal (heart rate coherence scores between 0.6-0.9, HRV in the ‘optimal’ range for the individual). The cognitive primitive extraction subsystem 176 may learn the physiological signature of flow for each individual and score real-time flow probability.

### Cognitive Load Assessment

Cognitive load (working memory demand, mental effort) is assessed through frontal theta power increases during high-load conditions, pupil diameter increases correlating with effort, response time variability (more variable when cognitively overloaded), and frontal-parietal alpha coherence changes. The cognitive primitive extraction subsystem 176 may produce real-time cognitive load estimates on a continuous scale, enabling detection of when task demands exceed current processing capacity.

### Biomarker Subsystem

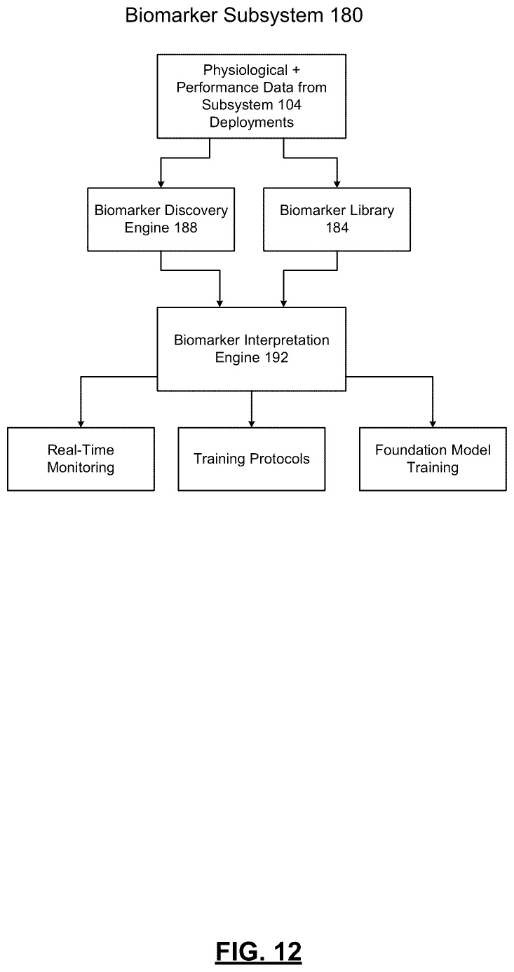

In various implementations, the platform 100 includes a biomarker subsystem 180, which is illustrated in FIG. 12. The biomarker subsystem 180 may include a biomarker library 184, a biomarker discovery engine 188, and a biomarker interpretation engine 192. For example, these represent the relationships between the physiology and the performance or neurotype. These biomarkers on their own are a data resource, which can be analyzed to identify new relationships, specifically in terms of neurotypes.

### A Priori Biomarker Library

The biomarker library 184 may store scientifically validated biomarkers. The biomarkers may include at least one of: Sensorimotor Rhythm (SMR) amplitude (12-15 Hz in motor cortex) that is elevated during motor preparation and correlates with movement accuracy, Frontal Midline Theta (4-7 Hz at Fz electrode) that increases during sustained attention tasks and is suppressed during distraction, Heart Rate Variability (HRV) metrics including RMSSD (reflecting parasympathetic tone) and HRV Coherence (reflecting optimal autonomic balance), Quiet Eye Duration (stable gaze on target) that predicts accuracy in targeting sports, and Alpha/Theta Ratio in frontal regions that increases during flow states.

For each biomarker, the biomarker library 184 may store at least one of: the physiological definition (frequency range, electrode location, computation method), performance correlations (strength and direction), individual normative ranges (95th percentile values for the population), or intervention recommendations (“if SMR amplitude drops below individual baseline, recommend motor preparation visualization”).

### Data-Driven Biomarker Discovery Engine

The biomarker discovery engine 188 may automatically identify novel physiological patterns correlated with performance. The process of automatically identifying novel physiological patterns correlated with performance includes unsupervised clustering analysis of foundation model embeddings from thousands of performance events, identifying recurrent physiological states by the biomarker discovery engine 188. For each state cluster, the biomarker discovery engine correlates the state cluster with performance outcomes (accuracy, speed, quality). If a state cluster shows strong performance correlation (e.g., state cluster X corresponds to 85% accuracy vs. 60% baseline), the biomarker discovery engine 188 extracts the physiological features defining that state. The biomarker discovery engine 188 validates across users and sessions (does this pattern generalize?). If validated, the biomarker discovery engine 188 adds it to the biomarker library 184 with an automatically generated interpretation (“State cluster 47 is characterized by elevated occipital alpha (11-13 Hz) and right parietal theta (5-7 Hz) and predicts 30% above-baseline performance; interpretation: strongly focused attention with some working memory load”).

The biomarker discovery engine 188 may identify novel biomarkers not found in the scientific literature, some of which are highly sport-specific (e.g., a pattern of right motor cortex beta desynchronization combined with stable eye fixation predicts successful basketball free throw execution with 92% accuracy).

### Biomarker Interpretation Engine

For each biomarker, the biomarker interpretation engine 192 may generate a natural language interpretation. In various implementations, the interpretations are generated by the LLM reasoning engine, grounding abstract biomarker values in cognitive terms meaningful to performers and coaches. An example interpretation includes: “Your frontal midline theta is currently 8.2 microvolts, which is 23% above your typical baseline. This indicates high sustained attention focus. Combined with your eye-tracking showing stable fixation on the target, your current cognitive state is optimally prepared for a demanding motor task.”

### Sport-Specific Implementations Of The Platform

### Basketball Free Throw Optimization

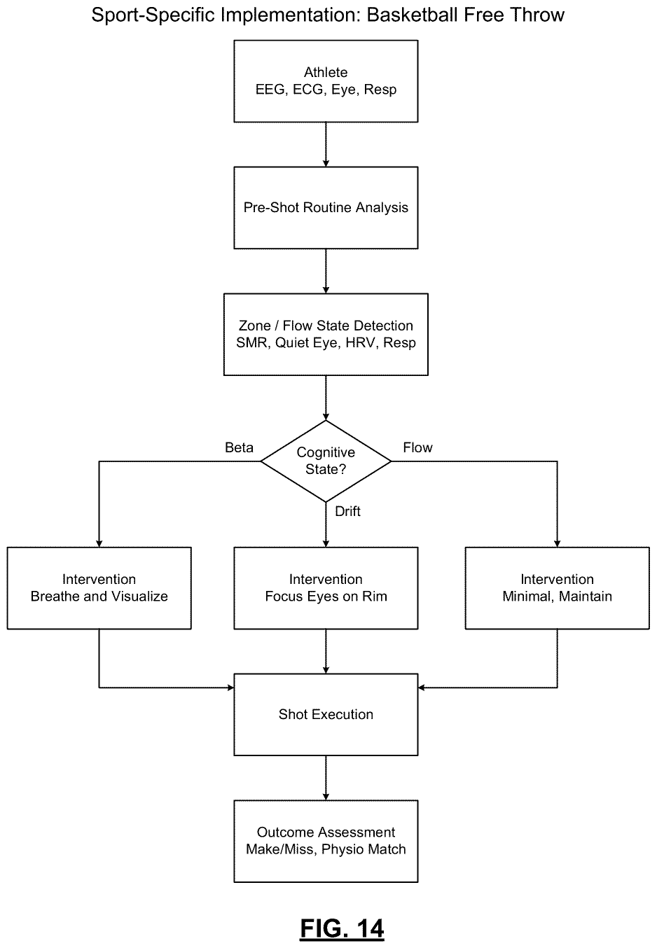

FIG. 14 illustrates a specific implementation of the platform 100 for a basketball player practicing shooting free throws. Basketball free throw performance may be predicted from the player's pre-shot physiological state with high accuracy. In various implementations, an optimal pre-shot physiological state is characterized by: (1) a pre-shot routine duration between 2.5-3.5 seconds (indicating that the player is neither rushed nor overthinking); (2) a heart rate variability coherence score greater than 0.7, indicating parasympathetic dominance and a calm state; (3) a sensorimotor rhythm amplitude above individual baseline; (4) a quiet eye duration exceeding 600 milliseconds, indicating focused visual attention on the rim; and (5) a respiratory rate between 4-6 breaths per minute, indicating slow controlled breathing.

Prior to the player attempting a shot, the platform 100 may detect deviations from an optimal physiological state (e.g., HRV coherence drops to 0.45, indicating elevated stress). The platform 100 may assess the probability that the current state will lead to successful execution of the shot attempt. This may be referred to as zone state detection. If the player's zone state is suboptimal, then an intervention protocol is triggered. In one example, if elevated beta indicates heightened arousal, the platform 100 delivers a haptic cue prompting the athlete to perform 4-7-8 breathing (4-second inhale, 7-second hold, 8-second exhale, which experimentally reduces beta oscillations by 35%). In another example, if eye-tracking shows attention drift from the rim, the platform 100 delivers a visual focus cue. If a flow state is detected (the player is in the optimal zone), the platform 100 minimizes intervention (in some cases, no intervention at all) and lets the athlete execute the shot attempt.

Data from basketball players using the above intervention protocol shows free throw accuracy improvements of 8-15% (in some cases, accuracy improves from ~70% to 76-81%), which is particularly pronounced in late-game high-pressure situations where stress-induced state deviations are most common.

### Multi-Sport Adaptability

The platform 100 also easily adapts to other sports besides basketball including: (1) Golf Putting—optimized for quiet eye duration (>1000 ms), smooth alpha oscillations, and minimal arousal fluctuation; (2) Baseball Batting—optimized for rapid attention shifts (pitch recognition), motor preparation timing, and reactive decision-making; (3) Soccer Penalty Kicks—optimized for stress inoculation (high-pressure state management), gaze behavior (visual focus on goalkeeper movement), and motor execution consistency; (4) Tennis Serving—optimized for rhythm (inter-serve interval consistency), arousal optimization (elevated but controlled), and proprioceptive feedback from the serve motion.

Each sport has sport-specific biomarkers identified through the discovery engine, sport-specific intervention protocols, and sport-specific performance metrics tracked.

### Cross-Sport Transfer Learning

In various implementations, the platform 100 develops hierarchical models that generalize from sport-specific patterns to domain-general cognitive skills. Techniques learned in basketball free throw training (stress inoculation, breathing protocols, attention control) transfer to other high-pressure performance contexts (soccer penalty kicks, baseball at-bats). The platform 100 may identify these transferable skills and recommend cross-training activities that reinforce common cognitive primitives across sports.

### Platform API Architecture

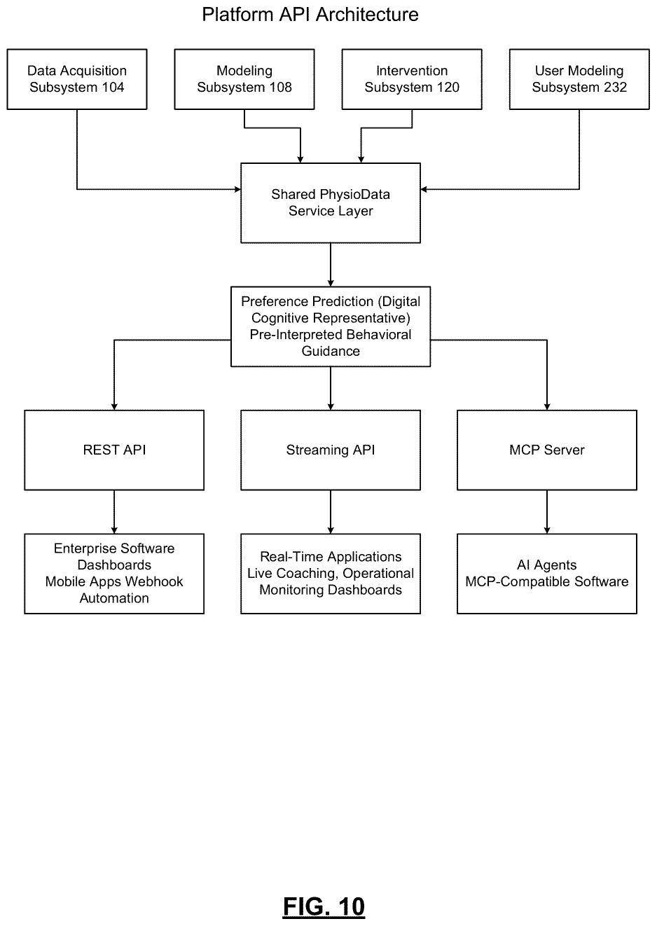

As shown in FIG. 10, in various implementations, the platform 100 exposes its physiological intelligence and capabilities through three complementary application programming interface (API) paradigms, enabling integration with external systems.

REST API: Standard HTTP endpoints enabling synchronous queries. Examples of queries include: /state/current (returns the current physiological state assessment and confidence), /predict/performance (predicts the next-outcome probability given the current state), /intervene/recommend (recommends the optimal intervention), /neurotype/profile (returns an individual's neurotype characterization), /biomarkers/active (returns the currently active biomarkers and their values). Responses are in JSON with structured and natural language components.

Streaming API: WebSocket or gRPC streaming providing real-time continuous updates. External applications subscribe to streams of real-time physiological state, performance predictions, intervention recommendations, or biomarker values. This enables live coaching dashboards, real-time performance monitoring systems, and integration with external coaching software.

Model Context Protocol (MCP): A structured tool interface protocol that enables LLM-based software agents to discover and invoke functions exposed by the platform 100. Through the structured interface protocol, external software agents can: query the operator's current cognitive state, request interruptibility assessments, obtain contextual help information, receive recommended interaction parameters, and report interaction outcomes. In various implementations, the structured interface protocol is implemented as an MCP server. Through MCP, external systems can also: request detailed analyses (“provide a detailed explanation of the physiological basis for this performer's current state”), query biomarker libraries, request model training on custom datasets, and integrate complex multi-step workflows combining platform capabilities with external systems.

In various implementations, the structured interface protocol 140 may be implemented as, without limitation: a Model Context Protocol (MCP) server, a REST API, a GraphQL API, a gRPC service, a WebSocket streaming interface, a command-line interface, a software development kit (SDK), a local inter-process communication channel, or any combination thereof. The structured interface protocol server may execute on any computing device, including the operator's local workstation, a remote cloud server, a mobile device, an edge computing node, a wearable device, or any other device capable of hosting the protocol, and may be discoverable by connected software agents via a local network, wide area network, or any other communication mechanism.

In various implementations, the platform 100 exposes pre-interpreted behavioral decision functions via the structured interface protocol rather than raw physiological signals, enabling consuming software agents to modify their behavior without requiring domain expertise in physiological signal interpretation. These behavioral decision functions include: an interruptibility assessment function that determines, given a specified urgency level, whether the software agent should engage the operator immediately or queue its output for later delivery; a contextual help function that provides the software agent with the operator's current error text, time spent in a stuck state, screen content summary, task category, and comprehension difficulty regions identified by gaze-content alignment; a response preferences function that returns recommended verbosity, complexity level, and presentation format based on the operator's current fatigue level, focus depth, and cognitive load; and an approach feedback function that infers whether the operator found a previous interaction helpful based on post-interaction behavioral signals.

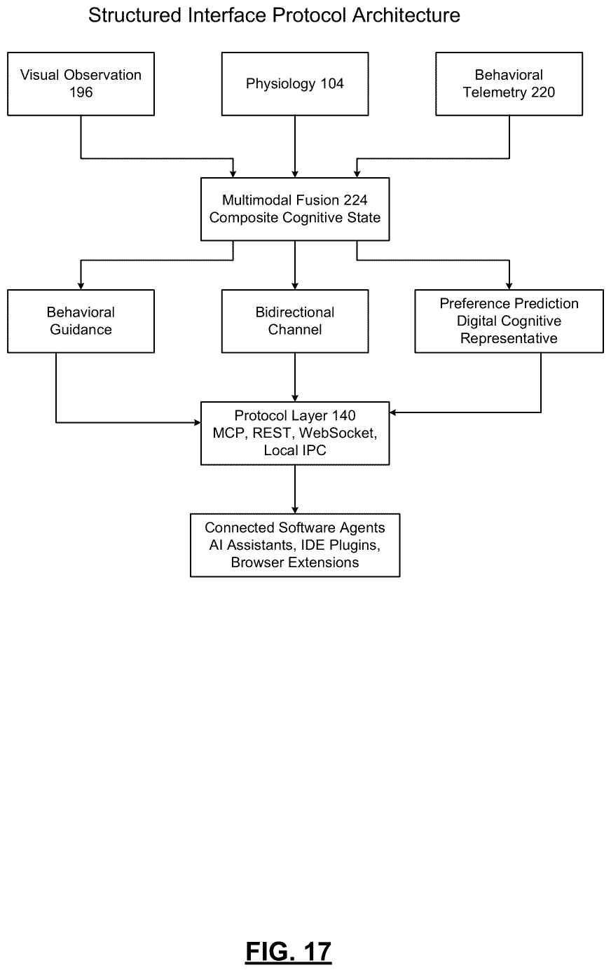

The structured interface protocol may further implement a bidirectional communication channel (FIG. 17) including: a pending output registration function whereby an external software agent registers a completed response with the platform 100, specifying a priority level and content summary, and the platform 100 queues the response for delivery at a moment when the operator's cognitive state permits interruption; and an outcome reporting function whereby an external software agent reports the result of a previous interaction, which the platform 100 incorporates into a policy that optimizes intervention selection and timing across the entire ecosystem of connected software agents.

The structured interface protocol may additionally expose a user preference prediction function that, given a set of options and a task context, returns a predicted operator preference with a confidence score, based on the operator's accumulated history of decisions, their contexts, their outcomes, and the operator's physiological and behavioral responses at decision points. This function enables connected software agents to act on the operator's behalf for routine decisions without requiring explicit operator input, effectively serving as the operator's digital cognitive representative.

The structured interface protocol may support multiple levels of abstraction for consuming applications with different levels of domain expertise. At the lowest level, raw physiological metrics (heart rate, pupil diameter, EEG frequency band power, heart rate variability) may be exposed to consuming applications with physiological signal processing expertise. At an intermediate level, derived cognitive state indicators (cognitive load estimate, arousal level, fatigue probability, attention quality score) may be exposed. At the highest level, pre-interpreted behavioral guidance (interruptibility assessment, recommended verbosity, contextual help content, approach feedback) may be exposed to consuming applications that require no domain expertise. Each connected software agent may query at whichever level of abstraction is appropriate for its capabilities.

All APIs enforce authentication (API keys, OAuth), rate limiting (to prevent abuse), and access control (restricting sensitive health information to authorized users). Data is encrypted in transit (TLS) and at rest (AES-256).

In various implementations, the platform API architecture supports two complementary levels of abstraction for exposing cognitive state intelligence. At the first level, the REST and Streaming APIs may expose raw and processed physiological metrics (heart rate, HRV, pupil diameter, EEG frequency band power, gaze coordinates) for applications that require direct access to physiological signals, such as research tools, clinical monitoring dashboards, and custom analytics pipelines. At the second level, the structured interface protocol may expose pre-interpreted behavioral guidance derived from the fusion of physiological, visual, and behavioral signals, abstracting away the underlying signal processing and presenting actionable intelligence (interruptibility assessment, contextual help, response preferences, approach feedback) for applications that require cognition-awareness without physiological expertise, such as AI coding assistants, communication tools, and productivity software. Both levels may be exposed simultaneously, enabling different consuming applications to access the appropriate level of abstraction for their use case.

### Time Scales and Data Flywheel Architecture

The platform 100 may operate on three timescales: (1) the real-time inference timescale (milliseconds to seconds) where raw sensor data is acquired, preprocessed, and embedded, and interventions are selected and delivered; (2) the session timescale (minutes to hours) where performance trends are analyzed, state evolution is visualized, and session-level insights are generated; and (3) the longitudinal timescale (days to months) where neurotyping profiles are refined, individual models are personalized, training programs are adapted, and biomarker libraries are expanded.

In various implementations, the platform 100 includes a data flywheel architecture that enables each performance session to contribute to the overall improvement of the platform 100. In the platform 100, raw sensor data flows into a foundation model, which extracts generalizable latent embeddings; performance outcomes are paired with these embeddings to improve downstream models; interventions and their outcomes are logged to train a policy optimization module; and novel physiological patterns are validated and added to the biomarker library. This creates positive feedback, enabling continuous improvement in prediction accuracy, intervention effectiveness, and neurotyping precision.

The data flywheel architecture ensures continuous system improvement. When a new athlete begins using the platform, their first sessions are analyzed using the global foundation model and personalized models trained on similar athletes. As the operator continues using the platform 100, performance and physiological data accumulate. Over time, personalized models are fine-tuned using the new athlete's data. If the athlete produces unusual physiological patterns, these are flagged for validation and potential addition to the biomarker library. The athlete's neurotyping is also refined as more sessions accumulate. Additionally, the intervention policy is updated based on this athlete's response patterns. Over weeks and months, the platform 100 becomes increasingly personalized, prediction accuracy increases, intervention effectiveness increases, and neurotyping precision increases.

For knowledge work applications, the data flywheel operates similarly: when a new knowledge worker begins using the platform, their initial sessions are analyzed using global models for task classification, stuck detection, and flow state identification; behavioral baselines for keyboard, pointing device, and gaze patterns are established from the first hours of use; stuck detection and intervention policies are personalized as the system observes which interventions the operator follows and which are dismissed; the user modeling subsystem accumulates the operator's decision patterns, work rhythms, and content engagement depth; and the visual observation subsystem builds an increasingly accurate model of the operator's typical workflows, error patterns, and productivity cycles.

The data flywheel operates equivalently for knowledge work deployments. When a new knowledge worker begins using the platform: (1) their initial sessions are analyzed using global models for task classification, stuck detection, and intervention selection; (2) behavioral telemetry baselines are established from keyboard, pointing device, and application usage patterns; (3) the visual observation subsystem learns the operator's typical applications, workflows, and error patterns; (4) the user modeling subsystem accumulates decision-outcome pairs and work pattern data; (5) intervention policies are personalized based on the operator's responsiveness to different intervention types and timing; (6) the episodic memory accumulates searchable records of work episodes. Over weeks and months, the system develops an increasingly accurate model of the operator's expertise, preferences, and cognitive patterns, enabling progressively more autonomous action on the operator's behalf.

The flywheel also improves global models. Data from thousands of users is aggregated (in a privacy-preserving fashion) to train improved foundation models, intervention policies, and prediction models. These improved global models benefit all users, while individual-specific adaptations preserve personalization.

For knowledge work applications, the data flywheel operates similarly: when a new knowledge worker begins using the platform 100, their initial sessions are analyzed using global models for task classification, stuck detection, and flow state identification; behavioral baselines for keyboard, pointing device, and gaze patterns are established from the first hours of use; stuck detection and intervention policies are personalized as the system observes which interventions the operator follows and which are dismissed; the user modeling subsystem 232 accumulates the operator's decision patterns, work rhythms, and content engagement depth; and the visual and behavioral observation subsystem 196 builds an increasingly accurate model of the operator's typical workflows, error patterns, and productivity cycles.

In various implementations, the platform 100 may maintain three interconnected data repositories. The first data repository is a raw physiological data lake storing all sensor recordings. In various implementations, the first data repository stores data gathered by the multimodal data acquisition subsystem 104. In various implementations, data is compressed before it is stored in the first data repository. In various implementations, data compression results in 1000 hours of recorded data across all modalities being compressed into 200 gigabytes (GB). A second data repository (sometimes referred to as an embedding cache) stores precomputed foundation model embeddings for efficient reanalysis. A third data repository (sometimes referred to as an analytics database) stores extracted features, predicted outcomes, intervention outcomes, and performance metrics.

### Cloud Infrastructure and Security

In various implementations, data ingestion pipelines within the platform 100 receive sensor data through Kafka or AWS Kinesis topics, distributing data to multiple processing streams. In various implementations, machine learning workloads within the platform 100 are orchestrated via Kubernetes, enabling automatic scaling based on computational demand. In various implementations, GPU-accelerated machine learning (NVIDIA GPUs) accelerates foundation model inference and policy optimization training. In various implementations, edge computing nodes deployed near the performer (on coaching tablets, wearable devices) perform low-latency inference for real-time feedback. In various implementations, latency-critical operations (intervention selection, real-time state estimation) run on an edge node, while higher-latency operations (model retraining, longitudinal analysis) run on cloud infrastructure.

In various implementations, the platform 100 uses TimescaleDB to store time-series data (physiological signals, performance metrics) and uses Apache Iceberg to store other data (raw sensor recordings, embeddings). In various implementations, federated learning protocols enable global model improvement while preserving user privacy, because users'data remains on their local servers, and only model gradients are shared with the central server, preventing raw data leakage.

In various implementations, the platform 100 encrypts all data in transit using TLS 1.3 and at rest using AES-256. In various implementations, user authentication for the platform 100 uses multi-factor authentication (MFA) with time-based one-time passwords. In various implementations, access control for the platform 100 follows role-based access control (RBAC) principles—performers can access only their own data, coaches can access their team's data, and researchers can access approved datasets with personally identifiable information (PII) removed. In various implementations, data anonymization and de-identification protocols ensure that research data cannot be linked back to individuals. In various implementations, the platform 100 is compliant with HIPAA (Health Insurance Portability and Accountability Act), GDPR (General Data Protection Regulation), and CCPA (California Consumer Privacy Act), enabling operation in regulated healthcare, European, and US markets.

### Conclusion

‘Physiological signals’ may include any biosignals reflecting the function of biological systems, including but not limited to neural activity (EEG), cardiac activity (ECG), autonomic activity (heart rate variability), ocular activity (eye-tracking, pupil diameter), respiratory activity, muscular activity (EMG), and kinematic data (motion capture, IMU accelerometry).

‘Large language model’ may refer to transformer-based neural network models trained on vast corpora of text data, including but not limited to GPT-4, Claude, Gemini, and other foundation models with contextual reasoning and natural language generation capabilities.

‘Embedding’ or ‘latent embedding’ may refer to a continuous-valued vector representation of data in a learned feature space, typically produced by neural networks, where semantic and structural relationships are preserved (similar data points have similar embeddings).

‘Visual capture’ may refer to any digital representation of the operator's task environment, including without limitation screenshots, video frames, augmented reality viewport captures, head-mounted display feeds, camera feeds from mobile devices, and any other visual representation of the environment in which the operator is performing a task.

‘Behavioral telemetry’ may refer to continuous streams of operator interaction data derived from input devices and development tools, including without limitation keyboard event patterns, pointing device dynamics, scroll behavior, version control activity, and application switching patterns.

‘Structured interface protocol’ may refer to any programmatic interface through which the system exposes cognitive state intelligence to external software agents, including without limitation Model Context Protocol (MCP) servers, REST APIs, GraphQL APIs, gRPC services, WebSocket interfaces, command-line interfaces, software development kits, and local inter-process communication channels.

‘Cognitive impasse’ or ‘stuck state’ may refer to a condition in which the operator is investing sustained behavioral effort without producing measurable task progress, as determined by the relationship between effort indicators and effectiveness indicators.

‘Operator’ and ‘user’ are used interchangeably throughout this specification to refer to any human individual whose cognitive performance is being assessed, predicted, or optimized by the system.

‘Digital cognitive representative’ may refer to the learned operator model maintained by the user modeling subsystem, which accumulates sufficient observations of the operator's decisions, preferences, and patterns to predict operator preferences and anticipate operator actions on behalf of connected software agents.

‘Visual capture’ may refer to any digital representation of the operator's task environment acquired by the system, including without limitation periodic screenshots of a computing display, continuous video streams, augmented reality or virtual reality viewport captures, head-mounted display feeds, camera feeds from mobile or wearable devices, and vehicle instrument panel captures.

‘Behavioral telemetry’ may refer to continuous streams of operator interaction data derived from input devices and software tools, including without limitation keyboard event patterns, pointing device dynamics, touchscreen interactions, version control activity, application switching sequences, and any other digitally observable operator interaction with the task environment.

‘Structured interface protocol’ may refer to a machine-readable communication interface through which the system exposes cognitive state intelligence to external software applications, including without limitation Model Context Protocol (MCP) servers, REST APIs, GraphQL APIs, gRPC services, WebSocket interfaces, command-line interfaces, software development kits, and local inter-process communication channels.

‘Cognitive impasse’ or ‘stuck state’ may refer to a condition in which the operator is investing behavioral effort in a task but producing insufficient measurable progress toward task completion, as determined by the relationship between effort indicators derived from behavioral telemetry and effectiveness indicators derived from visual observation of the task environment.

‘Digital cognitive representative’ may refer to the system's accumulated model of an operator's cognitive characteristics, preferences, decision patterns, and work history, which can be queried by external software agents through the structured interface protocol to obtain predicted operator preferences and anticipated operator actions, enabling the connected agents to act on the operator's behalf.

The above description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. In the written description and claims, one or more steps within a method may be executed in a different order (or concurrently) without altering the principles of the present disclosure. Similarly, one or more instructions stored in a non-transitory computer-readable medium may be executed in a different order (or concurrently) without altering the principles of the present disclosure. Unless indicated otherwise, numbering or other labeling of instructions or method steps is done for convenient reference, not to indicate a fixed order.

Numerical terms, such as “first,” “second,” and “third,” may be used in the disclosure and claims as unique labels: they are not used to imply a sequence or order unless the context clearly indicates otherwise. In other words, a “second” element could be relabeled as a “first” element without departing from the principles of the present disclosure. Further, the presence of a “second” element does not imply or require the presence of a “first” element. Similarly, the presence of a “first” element does not imply or require the presence of a “second” element.

Unless the context clearly indicates otherwise, the singular articles “a,” “an,” and “the” before a noun do not restrict the noun to a single instance. The verbs “comprise,” “include,” and “have” are inclusive and therefore specify the presence of elements without excluding the presence of one or more additional elements.

Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “coupled,” and “engaged.” Unless explicitly described as being “direct,” when a relationship between first and second elements is described in the above disclosure, that relationship encompasses a direct relationship where no other intervening elements are present between the first and second elements, as well as an indirect relationship where one or more intervening elements are present between the first and second elements.

The term “set” generally means a grouping of one or more elements. The elements of a set do not necessarily need to have any characteristics in common or otherwise belong together. However, in various implementations, a “set” may, in certain circumstances, be the empty set (in other words, the set has zero elements in those circumstances). As an example, a set of search results resulting from a query may, depending on the query, be the empty set. In contexts where it is not otherwise clear, the term “non-empty set” can be used to explicitly denote exclusion of the empty set—that is, a non-empty set will always have one or more elements.

A “subset” of a first set generally includes some of the elements of the first set. In various implementations, a subset of the first set is not necessarily a proper subset: in certain circumstances, the subset may be coextensive with (equal to) the first set (in other words, the subset may include the same elements as the first set). In contexts where it is not otherwise clear, the term “proper subset” can be used to explicitly denote that a subset of the first set must exclude at least one of the elements of the first set. Further, in various implementations, the term “subset” does not necessarily exclude the empty set. As an example, consider a set of candidates that was selected based on first criteria and a subset of the set of candidates that was selected based on second criteria; if no elements of the set of candidates met the second criteria, the subset may be the empty set. In contexts where it is not otherwise clear, the term “non-empty subset” can be used to explicitly denote exclusion of the empty set.

The phrase “at least one of A, B, and C” should be construed to mean a logical (A OR B OR C), using a non-exclusive logical OR, and should not be construed to mean “at least one of A, at least one of B, and at least one of C.” The phrase “at least one of A, B, or C” should be construed to mean a logical (A OR B OR C), using a non-exclusive logical OR. The phrase “A, B, and/or C” should be construed in the same way as the phrase “at least one of A, B, and C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgments of, the information to element A.

In this application, including the definitions below, the term “module” can be replaced with the term “controller” or the term “circuit.” In this application, the term “controller” can be replaced with the term “module.” The term “module” may refer to, be part of, or include processor hardware (shared, dedicated, or group) that executes code coupled with memory hardware (shared, dedicated, or group) that stores code executed by the processor hardware.

The module may include or be connected to one or more interface circuits configured to couple the module to one or more networks. An interface circuit may include, for example, one or more of ports, transceivers, antennas, physical-layer (PHY) circuitry, and medium access control (MAC) circuitry. Examples of networks include a wireless personal area network (WPAN), a local area network (LAN), a wireless local area network (WLAN), and a wide area network (WAN), including private networks, public networks, the Internet, and other Internet Protocol (IP)-based networks. Examples of WLAN technologies include IEEE 802.11 (sometimes referred to as Wi-Fi) wireless local area networking technology. Examples of wired LAN technologies include IEEE 802.3 (sometimes referred to as Ethernet) networking technology. Examples of WPAN technologies include IEEE 802.15.4-based protocols, such as Zigbee- or Thread-based communications, and Bluetooth-based protocols, including Bluetooth Classic and Bluetooth Low Energy (BLE), as specified by the Bluetooth SIG. Examples of wireless wide area network (WWAN) technologies include cellular technologies, such as Long-Term Evolution (LTE) and 5G New Radio (5G NR).

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a WAN such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as a remote, or cloud) module and a client (or user) module. For example, the client module may include a native or web application executing on a client device and in network communication with the server module.

The term “code,” as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

The memory hardware may also store data together with or separate from the code. Shared memory hardware encompasses a single memory device that stores some or all code from multiple modules. One example of shared memory hardware may be level 1 cache on or near a microprocessor die, which may store code from multiple modules. Another example of shared memory hardware may be persistent storage, such as a solid-state drive (SSD) or magnetic hard disk drive (HDD), which may store code from multiple modules. Group memory hardware encompasses a memory device that, in combination with other memory devices, stores some or all code from one or more modules. One example of group memory hardware is a storage area network (SAN), which may store code of a particular module across multiple physical devices. Another example of group memory hardware is random access memory of each of a set of servers that, in combination, store code of a particular module. The term “memory hardware” is a subset of the term “computer-readable medium.”

The apparatuses and methods described in this application may be partially or fully implemented by a special-purpose computer created by configuring a general-purpose computer to execute one or more particular functions embodied in computer programs. Such apparatuses and methods may be described as computerized or computer-implemented apparatuses and methods. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special-purpose computer, device drivers that interact with particular devices of the special-purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

The term non-transitory computer-readable medium does not encompass transitory electrical or electromagnetic signals propagating through a medium (such as on a carrier wave). Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD or DVD).

### CLAUSES

Various example embodiments of the invention are described in the following clauses.

Clause 1: A system for real-time performance assessment, prediction, and optimization comprising: (a) a multimodal physiological signal acquisition subsystem configured to simultaneously obtain electroencephalography (EEG), electrocardiography (ECG), eye-tracking, respiratory, and biomechanical signals from a human subject during task performance; (b) a neuroperformance foundation model that processes raw multimodal signals through self-supervised learning to produce generalizable latent state embeddings; (c) an embedding projection layer (Vector-ICL) that maps physiological embeddings into large language model token space; (d) a context-aware large language model reasoning engine configured to generate real-time state assessments, performance predictions, and intervention recommendations; and (e) a closed-loop intervention subsystem configured to select and deliver interventions based on real-time physiological state.

Clause 2: The system of clause 1, wherein the foundation model is trained using self-supervised learning objectives, including temporal contrastive learning, multimodal contrastive learning, masked signal reconstruction, and temporal prediction.

Clause 3: The system of any of clauses 1-2, wherein the LLM reasoning engine operates on mixed-modality inputs comprising both discrete tokens (task context, performance history) and continuous physiological embeddings interleaved in the input sequence.

Clause 4: The system of any of clauses 1-3, wherein the closed-loop intervention subsystem employs hierarchical reinforcement learning to learn intervention selection policies, with a high-level policy selecting intervention classes (breathing exercise, attention refocusing, motor preparation cue) and a low-level policy selecting intervention parameters.

Clause 5: The system of any of clauses 1-4, wherein the intervention selection policy models individual-specific intervention compliance probability, optimizing for expected performance improvement, accounting for both intervention efficacy and individual compliance likelihood.

Clause 6: The system of any of clauses 1-5, wherein the intervention selection policy enforces safety constraints, preventing the recommendation of interventions contraindicated in specific states (e.g., distraction-based interventions are not recommended during flow states).

Clause 7: The system of any of clauses 1-6, wherein intervention selection uses trajectory-aware policy optimization, accounting for multi-step physiological dynamics, simulating intervention sequences forward in time, and selecting sequences that optimize long-horizon performance outcomes.

Clause 8: The system of any of clauses 1-7, further comprising an AI Judge evaluation subsystem configured to assess decision quality across multiple dimensions, including information utilization, reasoning quality, timeliness, risk awareness, and outcome quality.

Clause 9: The system of any of clauses 1-8, further comprising a neurotyping subsystem configured to characterize individual-specific physiological response patterns and identify neurotype archetypes that determine personalized optimization strategies.

Clause 10: The system of any of clauses 1-9, further comprising reinforcement learning from human feedback (RLHF) training of the LLM reasoning engine, using expert evaluations of state assessments and intervention recommendations to align model outputs with expert judgment.

Clause 11: A method for real-time performance assessment and optimization, comprising: (a) acquiring multimodal physiological signals (EEG, ECG, eye-tracking, respiratory, biomechanical) from a subject during task performance; (b) preprocessing and removing artifacts from the signals; (c) processing the signals through a foundation model to generate physiological state embeddings; (d) projecting the embeddings into large language model token space; (e) querying a large language model with the projected embeddings and task context to generate state assessments and performance predictions; and (f) using the predictions to select and deliver real-time interventions.

Clause 12: The method of clause 11, wherein preprocessing includes independent component analysis (ICA) for artifact removal, including eye movement artifacts, muscle noise, cardiac artifacts, and movement artifacts.

Clause 13: The method of any of clauses 11-12, wherein the LLM generates state assessments through chain-of-thought prompting, first identifying dominant frequency bands and autonomic state, then assessing attention allocation from eye-tracking, and then synthesizing into an integrated state assessment.

Clause 14: The method of any of clauses 11-13, wherein performance predictions include the prediction of error probability in the next performance trial, the prediction of state evolution over the next 5 minutes, and the prediction of intervention responsiveness.

Clause 15: The method of any of clauses 11-14, wherein intervention selection uses contextual bandits with Thompson sampling to balance the exploitation of known-effective interventions with the exploration of potentially effective interventions.

Clause 16: The method of any of clauses 11-15, further comprising training the intervention policy using reinforcement learning signals derived from subsequent performance outcomes and intervention compliance.

Clause 17: The method of any of clauses 11-16, wherein the system supports interactive natural language queries, enabling users to ask questions about performance, receive detailed causal analyses, and explore physiological state-outcome relationships.

Clause 18: A method for individual neurotyping comprising: (a) acquiring physiological signals across multiple sessions from a subject; (b) extracting features from physiological embeddings, including baseline EEG frequency profiles, heart rate variability patterns, eye-tracking behavior, and response characteristics; (c) clustering features across subjects to identify neurotype archetypes; and (d) classifying the subject as a neurotype archetype, determining personalized optimization strategies, including training time-of-day, neurofeedback protocols, and intervention selection parameters.

Clause 19: The method of clause 18, wherein the system recommends circadian-based training schedules based on the subject's chronotype and time-of-day variation in physiological state quality.

Clause 20: The method of clauses 18 or 19, wherein neurofeedback protocols are personalized, directing Analytical Processor neurotypes to reduce task-related theta and increase alpha stability, directing Intuitive Performer neurotypes to enhance beta coherence and motor cortex engagement, and directing Pressure Responder neurotypes toward parasympathetic activation.

Clause 21: A method for scalable performance evaluation comprising: (a) training a large language model (the AI Judge) on expert human evaluations of decision quality across multiple dimensions (information utilization, reasoning quality, timeliness, risk awareness, outcome quality); (b) deploying the AI Judge to automatically evaluate decisions in real-time; (c) generating multidimensional quality scores and natural language justifications; and (d) using these evaluations as training targets for performance prediction models.

Clause 22: A platform application programming interface (API) system for exposing physiological intelligence comprising: (a) REST API endpoints enabling synchronous queries (/state/current, /predict/performance, /intervene/recommend, /neurotype/profile, /biomarkers/active); (b) a Streaming API providing real-time continuous updates via WebSocket or gRPC; (c) a Model Context Protocol (MCP) providing a structured tool interface enabling software agents to discover and invoke cognitive state assessment functions; and (d) authentication, rate limiting, and access control protecting sensitive health information.

Clause 23: The system of clause 22, wherein REST API responses are structured with both quantitative metrics and natural language explanations of physiological state, predictions, and recommendations.

Clause 24: The system of clauses 22 or 23, wherein the Streaming API enables external coaching dashboards and performance monitoring systems to receive real-time updates of physiological state, performance predictions, intervention recommendations, and biomarker values.

Clause 25: The system of any of clauses 22-24, wherein MCP exposes structured tool functions enabling external software agents to query cognitive state assessments, request interruptibility evaluations, obtain contextual help information, receive recommended interaction parameters, and report interaction outcomes for cross-agent optimization.

Clause 26: The system of clause 1, further comprising a visualization and user interface subsystem comprising: (a) a visualization engine configured to generate real-time performance visualizations, including at least line plots, heatmaps, and trend analyses with interactive data exploration capabilities; (b) a performance analysis engine configured to correlate physiological states with performance metrics, identify performance bottlenecks, and generate optimization recommendations; and (c) a web-based user interface providing real-time metrics display, interactive controls, and data export capabilities.

Clause 27: A method comprising: obtaining sensor data from a set of sensors equipped on an operator during performance of a first task, wherein the sensor data includes physiological data and behavioral data; obtaining environmental data associated with the first task; obtaining performance data associated with the operator performing the first task; correlating, in real-time, the sensor data with the environmental data and the performance data; and outputting, to a user interface, at least one of: a recommendation to optimize the performance of the first task, an identification of a bottleneck associated with the first task, or an insight into the operator's performance of the first task.

Clause 28: The system of clause 1, further comprising a cognitive primitive extraction module configured to identify fundamental cognitive building blocks from the multimodal physiological signals, the cognitive primitives comprising at least: focused visual attention measured through alpha power modulation in occipital regions and eye tracking fixation stability; motor preparation indicated by beta desynchronization patterns in motor cortex; flow state characterized by specific theta/alpha ratios in frontal regions and reduced default mode network activity; and cognitive load assessed through frontal theta power and pupil diameter variations.

Clause 29: The system of clause 1, further comprising a biomarker library and discovery subsystem comprising: (a) an a priori biomarker library storing scientifically validated physiological markers, including sensorimotor rhythm amplitude, frontal midline theta power, heart rate variability metrics, and quiet eye duration; (b) a data-driven biomarker discovery engine configured to automatically identify novel physiological patterns correlated with performance through unsupervised clustering and cross-session validation; and (c) a biomarker interpretation engine configured to generate natural language descriptions and relevance scores for each biomarker.

Clause 30: The system of clause 1, further comprising a multi-timescale feedback delivery system providing: (a) immediate feedback with latency less than 500 milliseconds delivered through at least one of haptic vibration, audio tones, and visual indicators on heads-up or augmented reality displays; (b) short-term feedback delivered after a predefined number of performance trials, comprising natural language coaching generated by the LLM and performance trend visualizations; (c) session-level feedback comprising comprehensive performance analysis and neurophysiological state evolution reporting; and (d) long-term adaptation comprising training plan modifications, neurotype profile updates, and predictive performance modeling.

Clause 31: A method for optimizing athletic performance in a specific sport, comprising: simultaneously acquiring multimodal physiological signals and behavioral data from an athlete during performance of a sporting activity; extracting cognitive primitives from the physiological signals, including focused visual attention, motor preparation state, and flow state indicators; detecting when the athlete's cognitive state is suboptimal for the specific sporting activity based on sport-specific cognitive state thresholds; generating and delivering a real-time intervention selected from a sport-specific intervention protocol; and adapting subsequent intervention selection based on the athlete's physiological response to delivered interventions.

Clause 32: The method of clause 31, applied to basketball free throw optimization, wherein: (a) the cognitive state thresholds comprise sensorimotor rhythm amplitude above an individual baseline, quiet eye duration exceeding 600 milliseconds, heart rate variability coherence score greater than 0.7, and respiratory rate between 4-6 breaths per minute; and (b) the sport-specific intervention protocol comprises: delivering a breathing and visualization cue when beta wave activity is elevated, delivering a visual focus cue when attention drift is detected, and minimizing intervention when flow state is detected.

Clause 33: The method of clause 31, further comprising: identifying sport-specific biomarker combinations for each supported sport; creating hierarchical models that generalize from sport-specific patterns to domain-general cognitive skills; and enabling cross-sport transfer of learned optimization strategies.

Clause 34: A cloud-based platform implementing the system of claim 1, comprising: distributed data collection nodes supporting multiple simultaneous users; GPU-accelerated machine learning infrastructure; federated learning capabilities preserving user privacy while improving global models; edge computing nodes for latency-critical feedback delivery; and comprehensive analytics dashboards for individual operators, athletes, and coaches.

Clause 35: The system of clause 1, further comprising predictive modeling capabilities to: forecast performance outcomes based on pre-activity physiological states; identify early indicators of performance degradation; recommend optimal training and competition schedules based on circadian neurophysiological patterns; and predict injury risk based on neurophysiological fatigue markers.

Clause 36: A system for real-time cognitive performance assessment, comprising: a visual capture module configured to acquire visual captures of an operator's task environment; a multimodal machine learning model configured to process the visual captures to extract task context including at least task category, application context, visible error states, and progress indicators; a multimodal integration module configured to combine the model-derived task assessments with at least one of physiological signals and behavioral telemetry to produce a composite cognitive performance assessment; and an intervention module configured to deliver performance-optimizing interventions to the operator based on the composite cognitive performance assessment.

Clause 37: The system of clause 36, wherein the multimodal machine learning model employs a hierarchical processing architecture comprising: a first processing stage that classifies task context at a lower computational cost; a set of trigger conditions that selectively invoke a deeper processing stage based on the first stage's outputs and at least one of detected state changes, temporal conditions, and behavioral signals, the trigger conditions being evaluated without invoking the deeper processing stage such that computational cost scales with the frequency of cognitively significant events rather than with elapsed time; and the deeper processing stage that produces performance assessments conditioned on the first stage's outputs and additional context.

Clause 38: The system of any of clauses 36-37, further comprising a gaze-content alignment module configured to: receive gaze fixation coordinates from an eye-tracking sensor; map the gaze fixation coordinates onto semantic content regions of the visual capture identified by the multimodal machine learning model; determine whether the operator's visual attention is directed at task-relevant content; detect persistent gaze regressions on a specific region of the visual capture exceeding a configurable duration threshold as a comprehension difficulty signal; identify the content of the regression region using the multimodal machine learning model; and trigger a contextual intervention specific to the identified content.

Clause 39: The system of any of clauses 36-38, further comprising a behavioral telemetry module configured to acquire continuous streams of operator interaction data, including at least keyboard interaction patterns, pointing device dynamics, and optionally version control activity, the behavioral telemetry being synchronized with physiological and visual observation channels via a common timing infrastructure and processed to produce temporal characterizations of operator interaction behavior.

Clause 40: The system of any of clauses 36-39, wherein the composite cognitive performance assessment comprises detection of a plurality of cognitive states, including at least: a stuck state characterized by absence of task progress combined with sustained effort indicators; a flow state characterized by a combination of indicators, including sustained focused engagement, consistent productive output, and absence of error escalation, among other indicators; and a comprehension difficulty state characterized by persistent attentional return to a specific content region of the visual capture, wherein the content of that region is identified by the multimodal machine learning model to enable content-specific intervention.

Clause 41: The system of any of clauses 36-40, wherein the system is further configured to predict cognitive impasse by: computing an effort indicator from behavioral telemetry, including keyboard activity, pointing device activity, and gaze engagement; computing an effectiveness indicator from visible progress detected via the multimodal machine learning model observation of visual captures, including content changes, error resolution, and work product advancement; computing a relationship between the effort indicator and the effectiveness indicator; and determining that the operator is in or approaching a stuck state when the effort indicator exceeds a first threshold and the effectiveness indicator falls below a second threshold, indicating high behavioral investment with low output quality.

Clause 42: A system for providing pre-interpreted cognitive state intelligence to external software agents via a structured interface protocol, comprising: a cognitive state assessment module that combines at least two of visual task observation, physiological signals, and behavioral telemetry to determine an operator's current cognitive state; and an interface layer that translates the cognitive state assessment into behavioral guidance for requesting software agents, the behavioral guidance comprising at least: an assessment of whether the operator's current cognitive state permits interruption by the requesting agent; contextual information relevant to the operator's current task, difficulties, and level of expertise; recommended interaction parameters for how the requesting agent should present information to the operator given the operator's current state; and an inference of whether the operator is satisfied with a previous interaction from the requesting agent, derived from observed post-interaction behavioral signals.

Clause 43: The system of clause 42, further comprising a bidirectional communication channel via the structured interface protocol, comprising: a pending output registration function whereby an external software agent registers a completed response with a priority level and content summary, and the system queues the response for delivery when the operator's cognitive state permits interruption; and an outcome reporting function whereby an external software agent reports the result of a previous interaction, which the system incorporates into a policy that optimizes intervention selection and timing across a plurality of connected software agents.

Clause 44: The system of any of clauses 42-43, further comprising a user preference prediction function that, given a set of options and a task context, returns a predicted operator preference with a confidence score based on accumulated decision history, decision contexts, outcomes, and physiological and behavioral responses at decision points, enabling connected software agents to act on the operator's behalf without requiring explicit operator input.

Clause 45: The system of clause 1, further comprising: a visual capture module configured to acquire visual captures of the operator's task environment; a multimodal machine learning model configured to process the visual captures to extract task context; a gaze-content alignment module configured to map gaze fixation coordinates onto semantic content regions of the visual capture; a behavioral telemetry module configured to acquire operator interaction data; and a multimodal fusion module configured to combine the physiological state embeddings from the neuroperformance foundation model with the visual task assessments, gaze-content alignment data, and behavioral telemetry to produce a composite cognitive performance assessment that integrates both physiological state and task context.

Clause 46: The system of clause 36, wherein operational deployment of the system generates labeled training data comprising visual captures paired with behavioral telemetry, audio captures, cognitive state assessments, and intervention outcomes, the labeled training data being used to improve the multimodal machine learning model, creating a compounding feedback loop wherein increased deployment generates improved model performance, which in turn generates higher-quality training data.

Clause 47: A method for detecting cognitive impasse in an operator performing a task, comprising: acquiring visual captures of the operator's task environment; processing the visual captures through a multimodal machine learning model to detect visible progress indicators, including content changes, error states, and work product advancement; acquiring behavioral telemetry, including at least keyboard interaction patterns and pointing device dynamics; computing an effort indicator from the behavioral telemetry, reflecting the degree of active behavioral investment; computing an effectiveness indicator from the visible progress indicators, reflecting the degree to which the operator's effort is producing measurable task advancement; and determining that the operator is in or approaching a cognitive impasse when the effort indicator exceeds a first threshold and the effectiveness indicator falls below a second threshold over a configurable temporal window.

Clause 48: The system of clause 36, wherein the visual capture module further captures facial images of the operator and processes them through an action unit analysis module that evaluates facial action units according to a configurable rubric to assess at least one of operator fatigue, postural indicators, and affective state, the action unit assessments being integrated into the composite cognitive performance assessment.

Clause 49: The system of clause 36, further comprising a user modeling subsystem configured to: accumulate observations of the operator's decisions, task contexts, outcomes, behavioral patterns, and physiological responses over a plurality of sessions; maintain vector representations of documents, artifacts, and content the operator has engaged with; learn the operator's decision policy, work patterns, cognitive preferences, and intervention responsiveness from the accumulated observations; and expose the learned operator model through a structured interface protocol, enabling connected software agents to query the system for predicted operator preferences and anticipated operator actions, such that the system serves as the operator's digital cognitive representative across the ecosystem of connected software agents.

Clause 50: The system of clause 49, wherein the user modeling subsystem is further configured to predict the operator's next action based on the current task context, recent behavioral trajectory, accumulated decision history, and current cognitive state, and to provide the predicted action to a requesting software agent with a confidence score, enabling the software agent to proactively execute or suggest the predicted action without requiring explicit operator input.

Clause 51: The system of clause 50, wherein the predicted next action is provided at a plurality of granularity levels, comprising: a high-level prediction of the next task type or application the operator is likely to engage; a mid-level prediction of the next operation within the current application; and a low-level prediction of the next input sequence or navigation target; each prediction accompanied by a confidence score; wherein low-confidence predictions are suppressed and high-confidence predictions are offered as suggestions, or, with operator consent, executed proactively by a connected software agent.

Clause 52: The system of clause 49, further comprising an episodic memory module configured to: store structured records of operator work episodes comprising task narratives, problems encountered, solutions applied, and decisions made with their outcomes; associate each episode with temporal context, cognitive state indicators, and vector embeddings enabling semantic retrieval; support natural-language querying wherein the operator or a connected software agent retrieves contextually relevant prior episodes via semantic similarity search; and proactively surface relevant prior context when the operator returns to a previously encountered task.

Clause 53: The system of clause 49, further comprising a semantic knowledge model configured to: build a persistent representation of the operator's domain expertise and content familiarity from gaze-content alignment data; distinguish content the operator scanned briefly from content studied carefully based on fixation duration, regression frequency, and re-reading patterns; and maintain a knowledge landscape mapping topics of deep expertise, areas of growing familiarity, and domains of persistent difficulty, enabling increasingly accurate expertise estimation and comprehension difficulty prediction over time.

Clause 54: The system of clause 36, further comprising an audio capture module configured to acquire speech and ambient sound from the operator's environment for at least one of: voice-based interaction with the system, auditory attention assessment through cortical speech tracking when combined with electroencephalography data, speaker identification and diarization for collaboration context detection, and speech content analysis for task context enrichment.

Clause 55: The system of clause 36, further comprising a privacy-preserving embedding extraction module configured to generate on-device, non-reconstructable representations of visual data for model training, comprising: an encoder that produces fixed-dimensional embedding vectors from visual captures of the operator's task environment; and a facial representation module that produces structural or geometric representations of facial images; wherein the embedding vectors and facial representations are semantically rich representations suitable for model training but cannot be used to reconstruct the original images or identify the operator.

Clause 56: A system for cognition-aware performance optimization, comprising: a multimodal sensing subsystem configured to acquire at least physiological signals from the operator and visual captures of the operator's task environment; a cognitive state assessment pipeline configured to process the multimodal signals through at least one of a neuroperformance foundation model and a multimodal machine learning model to produce composite cognitive performance assessments integrating physiological state with task context; a user modeling subsystem configured to accumulate observations of the operator's decisions, behavioral patterns, and physiological responses over a plurality of sessions and to learn the operator's preferences and work patterns from the accumulated observations; a closed-loop intervention subsystem configured to select and deliver performance-optimizing interventions based on the composite cognitive performance assessments; and a structured interface protocol configured to expose the cognitive performance assessments, the learned operator model, and pre-interpreted behavioral guidance to external software agents, enabling the external software agents to modify their interaction behavior based on the operator's current cognitive state without requiring domain expertise in physiological signal interpretation.

Clause 57: The system of clause 36, wherein the system is deployed in a local-first configuration wherein sensing, inference, and intervention delivery execute on the operator's device, the structured interface protocol is exposed as a local service accessible to other applications on the same device, and raw physiological data, visual captures, and behavioral telemetry are retained exclusively on the operator's device, with only derived scores, embeddings, or anonymized aggregate metrics optionally transmitted to remote infrastructure for model improvement.

Clause 58: The system of clause 36, wherein the multimodal integration module is configured to operate with any available subset of visual observation, physiological signals, and behavioral telemetry modalities, producing the most informative cognitive performance assessment given the available modalities, and wherein the system degrades gracefully when one or more modalities are unavailable.

Clause 59: The system of clause 36, wherein the multimodal machine learning model is further configured to detect decision points from visual captures, including moments when the operator selects between alternative approaches, commits to a course of action, accepts or rejects a suggestion from an external software agent, or responds to an external query, and wherein each detected decision point is recorded with its task context, the alternatives available, the action taken, the subsequent outcome, and the operator's physiological and behavioral state at the time of decision, enabling a user modeling subsystem to learn the operator's decision policy from accumulated decision-outcome pairs.

Clause 60: The system of clause 49, wherein the system is configured to respond to queries about the operator from connected software agents through the structured interface protocol, providing information about the operator's current task context, expertise level in specified domains, relevant work history, learned preferences, and anticipated needs, based on the accumulated operator model, such that the system represents the operator in interactions with software agents that the operator has not directly engaged, enabling connected software agents to personalize their behavior for the operator without requiring direct operator input or interaction.

Clause 61: A system for real-time cognitive performance assessment, comprising: a visual capture module configured to acquire visual captures of an operator's task environment; a multimodal machine learning model configured to process the visual captures to extract task context including at least task category, application context, visible error states, and progress indicators; a multimodal integration module configured to combine the model-derived task assessments with at least one of physiological signals and behavioral telemetry to produce a composite cognitive performance assessment; and an intervention module configured to deliver performance-optimizing interventions to the operator based on the composite cognitive performance assessment.

Clause 62: The system of clause 61, wherein the multimodal machine learning model employs a hierarchical processing architecture comprising: a first processing stage that classifies task context at a lower computational cost; a set of trigger conditions that selectively invoke a deeper processing stage such that computational cost scales with the frequency of cognitively significant events rather than with elapsed time; and the deeper processing stage that produces performance assessments conditioned on the first stage's outputs and additional context.

Clause 63: The system of clause 61, further comprising a gaze-content alignment module configured to receive gaze fixation coordinates from an eye-tracking sensor, map the gaze fixation coordinates onto semantic content regions of the visual capture identified by the multimodal machine learning model, and detect persistent gaze regressions exceeding a configurable duration threshold as a comprehension difficulty signal.

Clause 64: The system of clause 61, wherein the composite cognitive performance assessment comprises detection of at least: a stuck state characterized by absence of task progress combined with sustained effort indicators; and a flow state characterized by a combination of indicators including sustained focused engagement, consistent productive output, and absence of error escalation, among other indicators.

Clause 65: The system of clause 61, wherein the system is further configured to predict cognitive impasse by computing an effort indicator from behavioral telemetry and an effectiveness indicator from visible progress detected via the multimodal machine learning model and determining a stuck state when the effort indicator exceeds a first threshold and the effectiveness indicator falls below a second threshold.

Clause 66: A system for providing pre-interpreted cognitive state intelligence to external software agents via a structured interface protocol, comprising: a cognitive state assessment module that combines at least two of visual task observation, physiological signals, and behavioral telemetry to determine an operator's current cognitive state; and an interface layer that translates the cognitive state assessment into behavioral guidance for requesting software agents, the behavioral guidance comprising at least: an assessment of whether the operator's current cognitive state permits interruption; contextual information relevant to the operator's current task and difficulties; and an inference of whether the operator is satisfied with a previous interaction from the requesting agent.

Clause 67: The system of clause 66, further comprising a bidirectional communication channel comprising: a pending output registration function whereby an external software agent registers a completed response for delivery when the operator's cognitive state permits interruption; and an outcome reporting function whereby an external software agent reports the result of a previous interaction for cross-agent intervention optimization.

Clause 68: The system of clause 61, further comprising a user modeling subsystem configured to accumulate observations of the operator's decisions, task contexts, outcomes, and physiological responses over a plurality of sessions; maintain vector representations of content the operator has engaged with; and expose the learned operator model through a structured interface protocol enabling connected software agents to query the system for predicted operator preferences.

Clause 69: The system of clause 68, wherein the user modeling subsystem is further configured to predict the operator's next action based on the current task context, recent behavioral trajectory, and accumulated decision history, and to provide the predicted action to a requesting software agent with a confidence score.

Clause 70: The system of clause 61, wherein the system is deployed in a local-first configuration wherein sensing, inference, and intervention delivery execute on the operator's device, and raw physiological data, visual captures, and behavioral telemetry are retained exclusively on the operator's device.

Clause 71: A system for cognition-aware performance optimization, comprising: a multimodal sensing subsystem configured to acquire at least physiological signals from an operator and visual captures of the operator's task environment; a cognitive state assessment pipeline configured to process the multimodal signals to produce composite cognitive performance assessments integrating physiological state with task context; a user modeling subsystem configured to accumulate observations of the operator's decisions, behavioral patterns, and physiological responses over a plurality of sessions; a closed-loop intervention subsystem configured to select and deliver performance-optimizing interventions based on the composite cognitive performance assessments; and a structured interface protocol configured to expose the cognitive performance assessments and pre-interpreted behavioral guidance to external software agents.

Clause 72: A method for detecting cognitive impasse in an operator performing a task, comprising: acquiring visual captures of the operator's task environment; processing the visual captures through a multimodal machine learning model to detect visible progress indicators; acquiring behavioral telemetry including at least keyboard interaction patterns and pointing device dynamics; computing an effort indicator from the behavioral telemetry; computing an effectiveness indicator from the visible progress indicators; and determining that the operator is in or approaching a cognitive impasse when the effort indicator exceeds a first threshold and the effectiveness indicator falls below a second threshold.

Clause 73: The system of clause 61, wherein the multimodal integration module is configured to operate with any available subset of visual observation, physiological signals, and behavioral telemetry modalities, producing the most informative cognitive performance assessment given the available modalities.

Clause 74: A method comprising: obtaining sensor data from a set of physiological sensors associated with an operator during performance of a task; removing artifacts from the sensor data; converting the sensor data, using a foundation model, into a set of physiological state embeddings; projecting the set of physiological state embeddings into a token space of a large language model (LLM); generating an intervention for the operator, using the LLM, based on the set of physiological state embeddings and additional information about the task; and delivering the intervention to the operator.

Clause 75: The method of clause 74 wherein the sensor data includes at least one of: electroencephalography (EEG), electrocardiography (ECG), eye-tracking, respiratory monitoring, or biomechanical motion capture.

Clause 76: The method of clause 75 wherein obtaining the sensor data includes simultaneously obtaining EEG, ECG, eye-tracking, respiratory monitoring, and biomechanical motion capture data.

### Clause 77: the Method of Any of Clauses 74-76 Wherein the Foundation Model Is Trained

without using task-specific labels.

Clause 78: The method of any of clauses 74-77 wherein projecting the set of physiological state embeddings into the token space of the LLM is performed using a learned linear transformation or a shallow neural network.

Clause 79: The method of any of clauses 74-78 wherein the intervention is generated based on a structured prompt for the LLM that describes the task.

Clause 80: The method of any of clauses 74-79 wherein removing artifacts from the sensor data includes at least one of: high-pass filtering the sensor data, low-pass filtering the sensor data, notch filtering the sensor data, performing independent component analysis on the sensor data, visual artifact marking of the sensor data, or adaptive baseline correction of the sensor data.

Clause 81: The method of any of clauses 74-80 wherein the intervention is selected from a set of possible interventions.

Clause 82: The method of any of clauses 74-81 wherein delivering the intervention includes providing at least one of: haptic feedback, auditory feedback, visual feedback, or natural language coaching feedback to the operator.

Clause 83: The method of any of clauses 74-82 wherein the intervention is delivered within 500 milliseconds of the operator performing the task.

Clause 84: A system comprising: a set of physiological sensors configured to obtain sensor data of an operator during performance of a task; data processing hardware communicatively coupled to the set of physiological sensors and operable to: receive the sensor data; remove artifacts from the sensor data; convert, using a foundation model, the sensor data into a set of physiological state embeddings; project the set of physiological state embeddings into a token space of a large language model (LLM); generate, using the LLM, an intervention for the operator based on the set of physiological state embeddings and additional information about the task; and send the intervention to the operator; and a user device configured to receive the intervention and deliver the intervention to the operator.

Clause 85: The system of clause 84 wherein the sensor data includes at least one of: electroencephalography (EEG), electrocardiography (ECG), eye-tracking, respiratory monitoring, or biomechanical motion capture.

Clause 86: The system of clause 85 wherein obtaining the sensor data includes simultaneously obtaining EEG, ECG, eye-tracking, respiratory monitoring, and biomechanical motion capture data.

Clause 87: The system of any of clauses 84-86 wherein the foundation model is trained without using task-specific labels.

Clause 88: The system of any of clauses 84-87 wherein projecting the set of physiological state embeddings into the token space of the LLM is performed using a learned linear transformation or a shallow neural network.

Clause 89: The system of any of clauses 84-88 wherein the intervention is generated based on a structured prompt for the LLM that describes the task.

Clause 90: The system of any of clauses 84-89 wherein removing artifacts from the sensor data includes at least one of: high-pass filtering the sensor data, low-pass filtering the sensor data, notch filtering the sensor data, performing independent component analysis on the sensor data, visual artifact marking of the sensor data, or adaptive baseline correction of the sensor data.

Clause 91: The system of any of clauses 84-90 wherein the intervention is selected from a set of possible interventions.

Clause 92: The system of any of clauses 84-91 wherein delivering the intervention includes providing at least one of: haptic feedback, auditory feedback, visual feedback, or natural language coaching feedback to the operator.

Clause 93: The system of any of clauses 84-92 wherein the intervention is delivered within 500 milliseconds of the operator performing the task.

Clause 94: A closed-loop physiological control method for physiological monitoring and biofeedback delivery, the closed-loop physiological control method comprising: obtaining a set of physiological signals from a set of physiological sensors associated with an operator during performance of a task, wherein the set of physiological signals includes at least one of: electroencephalography (EEG) signals, electrocardiography (ECG) signals, eye-tracking signals, respiratory signals, or biomechanical motion signals; conditioning the set of physiological signals to remove artifacts, wherein conditioning the set of physiological signals includes at least one of: independent component analysis for removing eye movement artifacts and muscle noise, bandpass filtering, or notch filtering; processing the set of physiological signals through a first model to generate a set of physiological state embeddings; projecting the set of physiological state embeddings into a token space of a second model to produce a set of projected embeddings that can be interpreted by the second model; determining, using the second model operating on the set of projected embeddings and contextual information about the operator, a neurophysiological state of the operator, wherein the neurophysiological state includes at least one of: a cognitive load level, an attentional focus metric, an autonomic arousal level, or a motor preparation state; selecting, based on the neurophysiological state, a biofeedback stimulus from a set of biofeedback stimuli; and delivering the biofeedback stimulus to the operator through at least one of: a haptic feedback from a device worn by the operator, an auditory transducer, or a visual display element.

Clause 95: The closed-loop physiological control method of clause 94 wherein the first model is a foundation model.

Clause 96: The closed-loop physiological control method of any of clauses 94-95 wherein the second model is a large language model (LLM).

Clause 97: The closed-loop physiological control method of any of clauses 94-96 wherein the first model is a same model as the second model.

Clause 98: The closed-loop physiological control method of any of clauses 94-97 wherein obtaining the set of physiological signals includes simultaneously obtaining EEG, ECG, eye-tracking, respiratory monitoring, and biomechanical motion signals.

Clause 99: The closed-loop physiological control method of clause 95 wherein the foundation model is trained without using task-specific labels.

Clause 100: The closed-loop physiological control method of any of clauses 94-99 wherein projecting the set of physiological state embeddings into the token space of the second model is performed using a learned linear transformation or a shallow neural network.

Clause 101: The closed-loop physiological control method of any of clauses 94-100 wherein the biofeedback stimulus is generated based on a structured prompt for the second model that describes the task.

Clause 102: The closed-loop physiological control method of any of clauses 94-101 wherein delivering the biofeedback stimulus includes providing natural language coaching feedback to the operator.

Clause 103: The closed-loop physiological control method of any of clauses 94-102 wherein the biofeedback stimulus is delivered within 500 milliseconds of the operator performing the task.

Clause 104: A system for cognition-aware performance optimization, comprising: a multimodal sensing subsystem configured to acquire at least two modalities selected from physiological signals from the operator, visual captures of the operator's task environment, and behavioral telemetry from operator interactions with the system; a cognitive state assessment pipeline configured to process the acquired modalities through at least one of a neuroperformance foundation model and a multimodal machine learning model to produce composite cognitive performance assessments integrating physiological state, task context, or behavioral state; and an output subsystem configured to cause a performance-improving action based on the composite cognitive performance assessments, the performance-improving action comprising at least one of: (i) selecting and delivering a performance-optimizing intervention to the operator; or (ii) exposing the cognitive performance assessments and pre-interpreted behavioral guidance to an external software agent via a structured interface protocol, enabling the external software agent to modify its interaction behavior based on the operator's current cognitive state without requiring domain expertise in physiological signal interpretation.

Clause 105: A system for providing cognitive state intelligence to external software agents via a structured interface protocol, comprising: a cognitive state assessment module that combines at least two of visual task observation, physiological signals, and behavioral telemetry to determine an operator's current cognitive state; and an interface layer that translates the cognitive state assessment into behavioral guidance for requesting software agents, the behavioral guidance comprising at least one of: (i) an assessment of whether the operator's current cognitive state permits interruption by the requesting agent; (ii) contextual information relevant to at least one of: the operator's current task, the operator's difficulties, or the operator's level of expertise; (iii) recommended interaction parameters for how the requesting agent should present information to the operator given the operator's current state; or (iv) an inference of whether the operator is satisfied with a previous interaction from the requesting agent, derived from observed post-interaction behavioral signals.

Clause 106: A system for providing a digital cognitive representative of an operator to external software agents, comprising: a user modeling subsystem configured to: (a) accumulate, over a plurality of sessions, observations of the operator comprising at least one of: decisions, task contexts, outcomes, behavioral patterns, or physiological responses; (b) learn, from the accumulated observations, at least one of: the operator's decision policy, work patterns, cognitive preferences, or intervention responsiveness; and (c) expose the learned operator model through a structured interface protocol, enabling connected software agents to query the system for at least one of: predicted operator preferences or anticipated operator actions, such that the system serves as the operator's digital cognitive representative across the ecosystem of connected software agents.

Clause 107: The system of clause 104, wherein the multimodal machine learning model employs a hierarchical processing architecture comprising: a first processing stage that classifies task context at a lower computational cost; a set of trigger conditions that selectively invoke a deeper processing stage based on the first stage's outputs and at least one of detected state changes, temporal conditions, and behavioral signals, the trigger conditions being evaluated without invoking the deeper processing stage such that computational cost scales with the frequency of cognitively significant events rather than with elapsed time; and the deeper processing stage that produces performance assessments conditioned on the first stage's outputs and additional context.

Clause 108: The system of clause 104, further comprising a gaze-content alignment module configured to: receive gaze fixation coordinates from an eye-tracking sensor; map the gaze fixation coordinates onto semantic content regions of the visual capture identified by the multimodal machine learning model; determine whether the operator's visual attention is directed at task-relevant content; detect persistent gaze regressions on a specific region of the visual capture exceeding a configurable duration threshold as a comprehension difficulty signal; identify the content of the regression region using the multimodal machine learning model; and trigger a contextual intervention specific to the identified content.

Clause 109: The system of clause 104, wherein the composite cognitive performance assessments comprise detection of a plurality of cognitive states including at least: a stuck state characterized by absence of task progress combined with sustained effort indicators; a flow state characterized by a combination of indicators including sustained focused engagement, consistent productive output, and absence of error escalation; and a comprehension difficulty state characterized by persistent attentional return to a specific content region of the visual capture, wherein the content of that region is identified by the multimodal machine learning model to enable content-specific intervention.

Clause 110: The system of clause 104, wherein the system is further configured to predict cognitive impasse by: computing an effort indicator from behavioral telemetry including at least one of keyboard activity, pointing device activity, and gaze engagement; computing an effectiveness indicator from visible progress detected via the multimodal machine learning model observation of visual captures including at least one of content changes, error resolution, and work product advancement; computing a relationship between the effort indicator and the effectiveness indicator; and determining that the operator is in or approaching a stuck state when the effort indicator exceeds a first threshold and the effectiveness indicator falls below a second threshold, indicating high behavioral investment with low output quality.

Clause 111: The system of clause 104, wherein the multimodal sensing subsystem further captures facial images of the operator and processes them through an action unit analysis module that evaluates facial action units according to a configurable rubric to assess at least one of operator fatigue, postural indicators, and affective state, the action unit assessments being integrated into the composite cognitive performance assessments.

Clause 112: The system of clause 104, further comprising an audio capture module configured to acquire speech and ambient sound from the operator's environment for at least one of: voice-based interaction with the system, auditory attention assessment through cortical speech tracking when combined with electroencephalography data, speaker identification and diarization for collaboration context detection, or speech content analysis for task context enrichment.

Clause 113: The system of clause 104, further comprising a privacy-preserving embedding extraction module configured to generate on-device, non-reconstructable representations of visual data for model training, comprising: an encoder that produces fixed-dimensional embedding vectors from visual captures of the operator's task environment; and a facial representation module that produces structural or geometric representations of facial images; wherein the embedding vectors and facial representations are semantically rich representations suitable for model training but cannot be used to reconstruct the original images or identify the operator.

Clause 114: The system of clause 104, wherein the system is deployed in a local-first configuration wherein sensing, inference, and intervention delivery execute on the operator's device, the structured interface protocol is exposed as a local service accessible to other applications on the same device, and raw physiological data, visual captures, and behavioral telemetry are retained exclusively on the operator's device, with only derived scores, embeddings, or anonymized aggregate metrics optionally transmitted to remote infrastructure for model improvement.

Clause 115: The system of clause 104, wherein the cognitive state assessment pipeline is configured to operate with any available subset of the acquired modalities, producing the most informative composite cognitive performance assessment given the available modalities, and wherein the system degrades gracefully when one or more modalities are unavailable.

Clause 116: The system of clause 105, further comprising a bidirectional communication channel via the structured interface protocol, comprising: a pending output registration function whereby an external software agent registers a completed response with a priority level and content summary, and the system queues the response for delivery when the operator's cognitive state permits interruption; and an outcome reporting function whereby an external software agent reports the result of a previous interaction, which the system incorporates into a policy that optimizes intervention selection and timing across a plurality of connected software agents.

Clause 117: The system of clause 105, further comprising a user preference prediction function that, given a set of options and a task context, returns a predicted operator preference with a confidence score based on accumulated decision history, decision contexts, outcomes, and physiological and behavioral responses at decision points, enabling connected software agents to act on the operator's behalf without requiring explicit operator input.

Clause 118: The system of clause 106, wherein the user modeling subsystem is further configured to predict the operator's next action based on the current task context, recent behavioral trajectory, accumulated decision history, and current cognitive state, and to provide the predicted action to a requesting software agent with a confidence score, enabling the software agent to proactively execute or suggest the predicted action without requiring explicit operator input.

Clause 119: The system of clause 118, wherein the predicted next action is provided at a plurality of granularity levels comprising: a high-level prediction of the next task type or application the operator is likely to engage; a mid-level prediction of the next operation within the current application; and a low-level prediction of the next input sequence or navigation target; each prediction accompanied by a confidence score; wherein low-confidence predictions are suppressed and high-confidence predictions are offered as suggestions or, with operator consent, executed proactively by a connected software agent.

Clause 120: The system of clause 106, further comprising an episodic memory module configured to: store structured records of operator work episodes comprising task narratives, problems encountered, solutions applied, and decisions made with their outcomes; associate each episode with temporal context, cognitive state indicators, and vector embeddings enabling semantic retrieval; support natural-language querying wherein the operator or a connected software agent retrieves contextually relevant prior episodes via semantic similarity search; and proactively surface relevant prior context when the operator returns to a previously encountered task.

Clause 121: The system of clause 106, further comprising a semantic knowledge model configured to: build a persistent representation of the operator's domain expertise and content familiarity from gaze-content alignment data; distinguish content the operator scanned briefly from content studied carefully based on fixation duration, regression frequency, and re-reading patterns; and maintain a knowledge landscape mapping topics of deep expertise, areas of growing familiarity, and domains of persistent difficulty, enabling increasingly accurate expertise estimation and comprehension difficulty prediction over time.

Clause 122: The system of clause 106, wherein the user modeling subsystem is further configured to detect decision points from observations of the operator, including moments when the operator selects between alternative approaches, commits to a course of action, accepts or rejects a suggestion from an external software agent, or responds to an external query, and wherein each detected decision point is recorded with its task context, the alternatives available, the action taken, the subsequent outcome, and the operator's physiological and behavioral state at the time of decision.

Clause 123: The system of clause 106, wherein the system is configured to respond to queries about the operator from connected software agents through the structured interface protocol, providing information about the operator's current task context, expertise level in specified domains, relevant work history, learned preferences, and anticipated needs, based on the accumulated operator model, such that the system represents the operator in interactions with software agents that the operator has not directly engaged, enabling connected software agents to personalize their behavior for the operator without requiring direct operator input or interaction.

1. A closed-loop physiological control method for physiological monitoring and biofeedback delivery, the closed-loop physiological control method comprising:
obtaining a set of physiological signals from a set of physiological sensors associated with an operator during performance of a task, wherein the set of physiological signals includes at least one of: electroencephalography (EEG) signals, electrocardiography (ECG) signals, eye-tracking signals, respiratory signals, or biomechanical motion signals;
conditioning the set of physiological signals to remove artifacts, wherein conditioning the set of physiological signals includes at least one of: independent component analysis for removing eye movement artifacts and muscle noise, bandpass filtering, or notch filtering;
processing the set of physiological signals through a first model to generate a set of physiological state embeddings;
projecting the set of physiological state embeddings into a token space of a second model to produce a set of projected embeddings that can be interpreted by the second model;
determining, using the second model operating on the set of projected embeddings and contextual information about the operator, a neurophysiological state of the operator, wherein the neurophysiological state includes at least one of: a cognitive load level, an attentional focus metric, an autonomic arousal level, or a motor preparation state;
selecting, based on the neurophysiological state, a biofeedback stimulus from a set of biofeedback stimuli; and
delivering the biofeedback stimulus to the operator through at least one of: a haptic feedback from a device worn by the operator, an auditory transducer, or a visual display element.

2. The closed-loop physiological control method of claim 1 wherein the first model is a foundation model.

3. The closed-loop physiological control method of claim 2 wherein the foundation model is trained without using task-specific labels.

4. The closed-loop physiological control method of claim 1 wherein the second model is a large language model (LLM).

5. The closed-loop physiological control method of claim 1 wherein the first model is a same model as the second model.

6. The closed-loop physiological control method of claim 1 wherein obtaining the set of physiological signals includes simultaneously obtaining EEG, ECG, eye-tracking, respiratory monitoring, and biomechanical motion signals.

7. The closed-loop physiological control method of claim 1 wherein projecting the set of physiological state embeddings into the token space of the second model is performed using a learned linear transformation or a shallow neural network.

8. The closed-loop physiological control method of claim 1 wherein the biofeedback stimulus is generated based on a structured prompt for the second model that describes the task.

9. The closed-loop physiological control method of claim 1 wherein delivering the biofeedback stimulus includes providing natural language coaching feedback to the operator.

10. The closed-loop physiological control method of claim 1 wherein the biofeedback stimulus is delivered within 500 milliseconds of the operator performing the task.

11. A system comprising:
a set of physiological sensors configured to obtain sensor data of an operator during performance of a task;
data processing hardware communicatively coupled to the set of physiological sensors and configured to:
receive the sensor data;
remove artifacts from the sensor data;
convert, using a foundation model, the sensor data into a set of physiological state embeddings;
project the set of physiological state embeddings into a token space of a large language model (LLM);
generate, using the LLM, an intervention for the operator based on the set of physiological state embeddings and additional information about the task; and
send the intervention to the operator; and
a user device configured to receive the intervention and deliver the intervention to the operator.

12. The system of claim 11 wherein the sensor data includes at least one of: electroencephalography (EEG), electrocardiography (ECG), eye-tracking, respiratory monitoring, or biomechanical motion capture.

13. The system of claim 12 wherein obtaining the sensor data includes simultaneously obtaining EEG, ECG, eye-tracking, respiratory monitoring, and biomechanical motion capture data.

14. The system of claim 11 wherein the foundation model is trained without using task-specific labels.

15. The system of claim 11 wherein projecting the set of physiological state embeddings into the token space of the LLM is performed using a learned linear transformation or a shallow neural network.

16. The system of claim 11 wherein the intervention is generated based on a structured prompt for the LLM that describes the task.

17. The system of claim 11 wherein removing artifacts from the sensor data includes at least one of: high-pass filtering the sensor data, low-pass filtering the sensor data, notch filtering the sensor data, performing independent component analysis on the sensor data, visual artifact marking of the sensor data, or adaptive baseline correction of the sensor data.

18. The system of claim 11 wherein the intervention is selected from a set of possible interventions.

19. The system of claim 11 wherein delivering the intervention includes providing at least one of: haptic feedback, auditory feedback, visual feedback, or natural language coaching feedback to the operator.

20. The system of claim 11 wherein the intervention is delivered within 500 milliseconds of the operator performing the task.
